SEQUENCE LISTING (12 sequences)

SEQ ID NO: 1 - protein, 118 aa, Synthetic construct
QVQLQQSGAELARPGASVKMSCKASGYTFTRYMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 2 - protein, 15 aa, Synthetic construct
GGGGSGGGGSGGGGS

SEQ ID NO: 3 - protein, 107 aa, Synthetic construct
DIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 4 - protein, 214 aa, Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 450 aa, Synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 222 aa, Synthetic construct
QVQLQQSGAEVKKPGSSVKVSCKASGYTFTNYYIYWVRQAPGQGLEWIGGINPTSGGSNFNEKFKTRVTITADESSTTAYMELSSLRSEDTAFYFCTRQGLWFDSDGRGFDFWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVP

SEQ ID NO: 7 - protein, 219 aa, Synthetic construct
DIQMTQSPSSLSASVGDRVTITCRSSQNIVHSNGNTYLDWYQQTPGKAPKLLIYKVSNRFSGVPSRFSGSGSGTDFTFTISSLQPEDIATYYCFQYSHVPWTFGQGTKLQITREVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 214 aa, Synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 221 aa, Synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKC

SEQ ID NO: 10 - protein, 107 aa, Synthetic construct
KYLLPTAAAGLLLLAAQPAMAQPAEVQLVESGGGVVRPGGSLRLSCAASGFTFDDYGMSWVRQAPGKGLEWVSGINWNGGSTGYADSVRFTISRDNAKNSLYLQMNS

SEQ ID NO: 11 - protein, 140 aa, Synthetic construct
LRAEDTAVYYCARGRSLLFDYWGQGTLVTVSRSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVG

SEQ ID NO: 12 - protein, 11 aa, Synthetic construct
YGRKKRRQRRR

(19) United States
(12) Patent Application Publication
(10) Pub. No.: US 2025/0345455 A1
(43) Pub. Date: Nov. 13, 2025

(54) BISPECIFIC NANOPARTICLE BIOCONJUGATES

(71) Applicant: THE UNIVERSITY OF SYDNEY, New South Wales (AU)

(72) Inventors: Veysel KAYSER, New South Wales (AU); Mariam TARKISTANI, New South Wales (AU)

(73) Assignee: THE UNIVERSITY OF SYDNEY, New South Wales (AU)

(21) Appl. No.: 18/859,749

(22) PCT Filed: Apr. 26, 2023
(86) PCT No.: PCT/AU2023/050338

§ 371 Date: Oct. 24, 2024

(30) Foreign Application Priority Data
Apr. 26, 2022 (AU) 2022901088

(51) Int. Cl. A61K 47/69; A61K 47/68

(52) U.S. Cl. CPC A61K 47/6929; A61K 47/6849; A61K 47/6851

(57) ABSTRACT

The present invention relates to nanoparticle bioconjugates comprising binding proteins for binding to a cancer cell and an antigen of an immune cell, and uses and methods of treatment comprising administration thereof.

### FIELD OF THE INVENTION

The present invention relates to nanoparticle bioconjugates, compositions comprising the same and methods of treatment and uses thereof.

### CROSS-REFERENCE TO EARLIER APPLICATION

This application claims priority from Australian provisional application AU 2022901088, the contents of which are hereby incorporated by reference in their entirety.

### BACKGROUND OF THE INVENTION

Targeted immunotherapies using monoclonal antibodies (mAbs) have been extremely successful due to their unmatched specificities and efficacies. Their growth in the last two decades has been unprecedented with wide-ranging applications from oncology to inflammatory diseases. Recently, different types of antibody-based products including bispecific antibodies (biAbs) have emerged, targeting mainly different types of cancers. BiAbs can bind to both tumour cells and immune cells (e.g., killer T-cells) simultaneously so that the tumour cells are lysed precipitously. BiAbs have shown great therapeutic potential as emerging immunotherapies. Currently, two biAbs are approved as cancer therapies, and hundreds of new candidates are in clinical trials.

Currently available biAbs suffer from low conformational and formulation stabilities and have a short serum half-life leading to their rapid elimination from the body, e.g., blinatumomab has a half-life of 2.1 hours. Several hundreds of different biAb constructs have been reported so far, and most of these constructs suffer from stability and half-life issues. This represents a significant limitation for the use of these molecules for the treatment of cancer, where a longer serum half-life is essential for better therapeutic outcomes.

There is a need for improved bispecific binding molecules which have increased conformational and/or formulation stability.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a nanoparticle bioconjugate comprising:
a 1^{st} nanoparticle to which is conjugated a 1^{st} binding protein for specifically binding a target moiety on a cancer cell, and
a 2^{nd} nanoparticle, to which is conjugated a 2^{nd} binding protein for specifically binding a target moiety on an immune cell;
wherein the nanoparticles are arranged in a dumbbell or flower-like configuration, and
wherein the bioconjugate is capable of binding a cancer cell via the 1^{st} binding protein and an immune cell via the 2^{nd} binding protein.

In certain embodiments, the 1^{st} nanoparticle is a metallic nanoparticle and the 2^{nd} nanoparticle is a magnetic nanoparticle. In alternative embodiments, the 1^{st} nanoparticle is a magnetic nanoparticle and the 2^{nd} nanoparticle is a metallic nanoparticle. The metallic nanoparticle may be magnetic or non-magnetic.

Preferably, the architecture of the nanoparticle bioconjugate is in the form of a central metallic nanoparticle, surrounded by 3-4 or more magnetic nanoparticles, in a flower-like configuration.

Alternatively, the nanoparticle bioconjugate may be in the form of a central (core) magnetic nanoparticle, surrounded by 3-4 or more metallic nanoparticles, in a flower-like configuration.

In alternative embodiments, the nanoparticle bioconjugate may comprise the 1^{st} and 2^{nd} nanoparticles in a “dumbbell-like” architecture, whereby the nanoparticle conjugate comprises a metallic nanoparticle adjacent to, or in intimate contact, with a magnetic nanoparticle.

Accordingly, it will be understood that the nanoparticle bioconjugate of the invention is not in the form of a spherical nanoparticle.

In any embodiment of the invention, the magnetic nanoparticle may be selected from: an Fe₃O₄ (iron oxide) nanoparticle, an Fe(CO)₅ (iron pentacarbonyl) nanoparticle or an FeCl₂ (iron chloride) nanoparticle.

In any embodiment, the metallic nanoparticle may be selected from: a gold (Au), a silver (Ag), platinum (Pt) or palladium (Pd), copper (Cu), nickel (Ni), cobalt (Co), or an alloy of two or more thereof or nanoparticle.

Preferably, the metallic nanoparticle is a gold or silver nanoparticle. In particularly preferred embodiments, the metallic nanoparticle is a gold nanoparticle.

In any embodiment, the nanoparticle bioconjugate is comprised of iron and gold nanoparticles, preferably wherein the bioconjugate comprises a central gold nanoparticle surrounded by 3-4 or more iron nanoparticles to form a flower-shaped nanoparticle.

In any embodiment, the nanoparticle bioconjugate is comprised of iron and silver nanoparticles, preferably wherein the bioconjugate comprises a central silver nanoparticle surrounded by 3-4 or more iron nanoparticles to form a flower-shaped nanoparticle.

In any embodiment, the nanoparticle bioconjugates of the invention may be between about 5 nm to about 100 nm. In preferred embodiments, the nanoparticle bioconjugates may be between about 35 nm to 80 nm, preferably about 40 nm.

In any embodiment, the 1^{st} and 2^{nd} binding proteins are covalently conjugated to the 1^{st} and second nanoparticles. It will be appreciated that any conjugation method may be used in order to conjugate the 1^{st} and 2^{nd} binding proteins to the nanoparticles. Such methods are well known to the skilled person and in the art.

In any embodiment, the 1^{st} and 2^{nd} antigen binding proteins are covalently conjugated to the 1^{st} and 2^{nd} nanoparticles via different chemistries. It will be appreciated that conjugation methods will depend on the functional groups that are available and the surface modification of the nanoparticle.

In certain embodiments, the binding protein(s) may be directly conjugated to the nanoparticles. In alternative embodiments, the binding protein(s) may be conjugated via a linker moiety. The linker moiety may be any commercially available linker as further described herein or other linker for conjugating a peptide or protein moiety to a nanoparticle.

In certain examples, the binding protein may be covalently conjugated to the nanoparticle, preferably a functionalised gold nanoparticle, via a polyethylene glycol (PEG) moiety, an alkyne, an amine, an azide, biotin, a carboxyl, a hydroxyl, a methyl, maleimide, neutravidin, NHS, a hydrazone or a thiol linker group.

In certain examples, the binding protein may be covalently conjugated to the nanoparticle, preferably a magnetic nanoparticle such as iron, via an amine, an azide, a carboxyl, maleimide, or a hydrazone linker group.

In any embodiment, the thiol linker may be at least one of thioctic acid, monothioctic acid, dithioctic acid, and trithioctic acid.

The 1^{st} binding protein may be any binding protein capable of specifically binding to a target moiety of a cancer cell. The cancer cell may be any selected from: breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

In any embodiment, the target moiety on the cancer cell may be an antigen selected from: a sugar, a lipid, a nucleic acid, a peptide or a protein. Preferably the target moiety is endogenous to the cancer cell.

The binding protein for binding the target moiety may comprise, consist essentially of or consist of an antigen binding domain. In the alternative, the binding protein may comprise, consist essentially of, or consist of a receptor or ligand, or part thereof, for binding the cancer cell. In any aspect, the target or target molecule may be any one described herein.

Examples of cancer cell antigens that may be specifically bound by the 1^{st} antigen binding protein include: HER2, EGFR, mesothelin, PSMA, GPC3, MUC1, GD2, CEA, EpCAM, LeY, PCSA, CD19, CAIX, CD20, Clec9a, CD276, PD-L1 and PD-L2. Other examples of cancer cell antigens are further described herein.

In particularly preferred embodiments, the antigen is HER2 and the cancer cell is any cancer cell overexpressing HER2 or is HER2 positive. Accordingly, in preferred embodiments, the 1^{st} binding protein is for binding to HER2 on a HER2-positive cancer cell, such as breast cancer or stomach cancer.

Examples of HER2 antibodies and binding proteins derived therefrom are known in the art, and include pertuzumab and trastuzumab.

In alternative embodiments, the antigen is EGFR and the cancer cell is any cancer cell overexpressing epidermal growth factor receptor (EGFR). Accordingly, in alternative preferred embodiments, the 1^{st} binding protein is for binding EGFR on a EGFR-positive cancer cell, such as: a squamous cell carcinoma of head and neck (SCCHN), glioma, nasopharangeal cancer, or pancreatic cancer cell.

Examples of EGFR antibodies and binding proteins derived therefrom are known in the art, and include panitumumab and nimotuzumab.

In any embodiment of the invention, the 1^{st} antigen binding protein is selected from: trastuzumab, pertuzumab, panitumumab, nimotuzumab, cetuximab, or an antigen binding fragment thereof.

The 2^{nd} binding protein may be any binding protein capable of specifically binding to a target moiety of an immune cell. The immune cell may be any immune cell, optionally one selected from: a T cell, an antigen presenting cell (APC), or an natural killer (NK) cell. Preferably the immune cell is a T cell.

In any embodiment, the target moiety on the immune cell may be an antigen selected from: a sugar, a lipid, a nucleic acid, a peptide or a protein. In any embodiment, the target moiety on the immune cell may be an antigen selected from: a sugar, a lipid, a nucleic acid, a peptide or a protein. Preferably the target moiety is endogenous to the immune cell. The binding protein for binding the target moiety may comprise, consist essentially of or consist of an antigen binding domain. In the alternative, the binding protein may comprise, consist essentially of, or consist of a receptor or ligand, or part thereof, for binding the immune cell. In any aspect, the target or target molecule may be any one described herein.

In preferred embodiments of the invention, the immune cell is a T cell and the target moiety on the T cell that is bound by the 2^{nd} binding protein, is any one selected from: CD3, CD2, CD4, CD7, CD8, PD1, CTLA4, KIR, CD16, CD94, CD161, NTBA, CD19, CD20, CD22, CD30, CD33, CD38, CD40L, CD44, CD56, CD79b, CD80, CD86, CD135, CD137, CD138, CD154, EphA2, EGFR, and any combination thereof.

Accordingly, in preferred embodiments, the immune cell is a T cell and the 2^{nd} binding protein is selected from: an anti-CD3 antibody, an anti-CD2 antibody, anti-CD4 antibody, anti-CD7 antibody, anti-CD8 antibody, anti-PD1 antibody, anti-CTLA4 antibody, anti-KIR antibody, anti-CD16 antibody, anti-CD94 antibody, anti-CD161 antibody, anti-NTBA antibody, recombinant human NTBA, anti-CD19 antibody, anti-CD20 antibody, anti-CD22 antibody, anti-CD30 antibody, anti-CD33 antibody, anti-CD38 antibody, anti-CD40L antibody, anti-CD44 antibody, anti-CD56 antibody, anti-CD79b antibody, anti-CD80 antibody, anti-CD86 antibody, anti-CD135 antibody, anti-CD137 antibody, anti-CD138 antibody, anti-CD154 antibody, anti-EphA2 antibody, anti-EGFR antibody, or a fragment thereof.

In some embodiments of the invention, when the immune cells are T cells the binding protein may be an anti-CD3 antibody (e.g. OKT3, UCHT1, IP26, SK7, HIT3a or other clones) and/or an anti-CD4 antibody and/or an anti-CD8 antibody and/or an anti-PD1 antibody, and/or an anti-CTLA4 antibody and the like or a combination thereof.

If the immune cells are NK cells, the binding protein is preferably an anti-KIR antibody and/or an anti-CD16 antibody and/or an anti-CD94 antibody and/or an anti-CD161 antibody and/or an anti-CD56 antibody and the like or a combination thereof.

It will be understood that the 1^{st} and/or 2^{nd} binding proteins may be any suitable polypeptide capable of binding to an antigen. For example, the antigen binding protein may be an antibody, an antibody fragment, a genetically engineered antibody, a chimeric antibody, a heteroconjugate antibody, or a combination thereof.

In certain embodiments, the binding protein is an antibody. Optionally the antibody is selected from IgA, IgD, IgE, IgG, IgM. Preferably the antibody is an IgG. The IgG may be an IgG1, IgG2, IgG3, IgG4.

In any embodiment, the binding protein may be an antibody fragment, optionally selected from: a recombinant antibody fragment, a diabody, a triabody, a chimeric antibody, an F(ab′)2 fragment, an Fab′ fragment, an Fab′-SH fragment, a Fab fragment, an sFv fragment, a dsFv fragment, a bispecific sFv fragment, a bispecific dsFv fragment, a single chain Fv protein (scFv), a disulfide stabilized Fv protein, or a combination thereof.

In certain embodiments, the 1^{st} and 2^{nd} binding proteins may be of the same antibody format. For example, both binding proteins may be in the form of an IgG, or any other antibody format described herein including any antigen binding fragment of an IgG (such as an scFv, Fab′ or other). In alternative embodiments, the 1^{st} and 2^{nd} antigen binding proteins are different antibody formats. For example, in certain embodiments, the 1^{st} binding protein may be in the form of a monoclonal immunoglobulin (mAb, IgG) and the 2^{nd} binding protein may be in the form of an antibody fragment, such as an scFv. The 1^{st} and/or 2^{nd} binding proteins may be mono-specific or multispecific (such as bi-specific or tri-specific).

In further embodiments, the bioconjugate may further comprise a moiety for enabling cell penetration, such as a cell penetrating peptide (CPP). Examples of suitable cell penetrating peptides are known to the skilled person and non-limiting examples include peptides from the human immunodeficiency virus 1 (HIV1) Tat protein (eg Tat⁴⁸⁻⁶⁰ or Tat⁴⁷⁻⁵⁷; penetration (eg Antp⁴³⁻⁴⁸); transportan; oligoarginine (eg R₈); MAP; MPG; SAP (See, for example, Veldhoen et al. 2008 Int. J. Mol. Sci. 9 (7), 1276-1320, https://doi.org/10.3390/ijms9071276; incorporated by reference in its entirety). The CPP may be conjugated to the nanoparticle by via a suitable linker moiety, such as the linker moieties described herein for the conjugation of binding proteins.

In some embodiments, the bioconjugate further comprises a HIV-TAT cell penetrating peptide, preferably Tat⁴⁷⁻⁵⁷ (SEQ ID NO:12). The HIV-TAT CPP may be attached to the nanoparticle via a thiol linker, for example thiol-PEG-NHS (See eg Cruz and Kayser 2019, Cancers 116 (6), page 870, https://doi.org/10.3390/cancers11060870; incorporated by reference in its entirety).

The invention also provides for a pharmaceutical composition comprising any nanoparticle bioconjugate described herein, optionally in combination with a pharmaceutically acceptable excipient or carrier.

The present invention also provides a method of treating or preventing cancer in a subject, the method comprising administering to a subject in need thereof, a nanoparticle bioconjugate or pharmaceutical composition as described herein. As used herein, methods of treating cancer include methods of inhibiting, preventing or minimising spread or progression of a cancer, including inhibiting or preventing metastasis of cancer.

Further still, the invention provides for the use of a nanoparticle bioconjugate of the invention, in the manufacture of a medicament for the treatment or prevention of cancer.

The invention further provides a nanoparticle bioconjugate or a pharmaceutical composition of the invention for use in the treatment or prevention of cancer.

The invention further provides a kit for the generation of a nanoparticle bioconjugate of the invention. Preferably the kit comprises the nanoparticles, binding proteins and instructions for the combination thereof in order to obtain a bioconjugate capable of binding cancer cells and immune cells.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: A. Schematic depicting the conjugation of 1^{st} and 2^{nd} antigen binding proteins to nanoparticles, preferably in a flower-shaped arrangement, and use thereof to simultaneously target T cells and cancer cells. B. Thermal decomposition synthesis of magnetic gold nanoparticles. Iron and gold precursors are dissolved in an organic solvent, chemical precipitation is achieved at 310° C. under vigorous stirring in the presence of N₂ gas. After cooling, the nanoparticles were purified and stored.

FIG. 2: Characterization of Fe₃O₄@Au nanoparticles. A. Transmission electron microscopy (TEM) image of representative flower-like nanoparticles. (i) TEM of representative hydrophobic tail nanoparticle (Fe₃O₄@Au/OA). The diameters of the gold core and iron are about 11 and 14 nm respectively. (ii) TEM of representative hydrophilic tail nanoparticles with the dopamine layer (Fe₃O₄@Au/Dop). Average diameter of the shell is 4 nm. B. FTIR spectra of the hydrophobic Fe₃O₄@Au nanoparticles (upper line) and hydrophilic Fe₃O₄@Au nanoparticles (lower line). The wavenumbers were records from 600 to 4000 cm-1.

FIG. 3: MALDI-TOF mass analysis of Nmab-Fe₃O₄@Au-aCD3scFv NPs. Mass spectra of the Fe₃O₄@Au NPs before and after coupling with the anti-EGFR mAB (Nimotuzumab; 149 KDa), and anti-CD3 scFv (24 KDa).

FIG. 4: Normalized UV-Visible absorption spectroscopy of the modified nanoparticles: A. Hydrophobic Fe₃O₄@Au NPs showing its strong absorption at 573 nm while shifted to 390 nm after the modification. B. NmabNPs (light green) and BisNPs (red); showing the total amount of loaded antibodies; NmabNPs (blue); BisNPs (green); showing the very strong absorption of the conjugated antibodies after the centrifugation at around 260 nm.

FIG. 5: Nanodrop protein measurements of conjugated Fe₃O₄@Au NPs. A. PBS base line, B. Bare NPs negative control, C. Bis-Fe₃O₄@Au NPs (conjugated bispecific NPs), D. Fe₃O₄@Au-αCD3scFv NPs, E. Nmab-Fe₃O₄@Au NPs.

FIG. 6: Sodium dodecyl sulfate-polyacrylamide gel electrophoresis (SDS-PAGE) analysis of conjugated Fe₃O₄@Au NPs. Lane 1, protein size marker; Lane 2, free Nimotuzumab; Lane 3, blank control; Lane 4, anti-CD3 scFv; Lane 5, NPs; lane 6, Nmab-Fe₃O₄@Au NPs; Lane 7, Bis-Fe₃O₄@Au NPs (bispecific nanoparticles conjugated to nimotuzumab and anti-CD3 scFv; otherwise referred to as Nmab-Fe₃O₄@Au-αCD3scFv).

FIG. 7: Cytotoxicity assay of co-cultured A431 and Jurkat cells at ratio 1:5 (Target:Effector) with Fe₃O₄@Au nanoparticles (0-5000 μg/ml iron). The viability of co-cultured cells was not significantly affected until treated with Fe₃O₄@Au NPs with a Fe concentration >2500 μg/ml. Data are reported as means±SD.

FIG. 8: Cytotoxicity assay of co-cultured A431 and Jurkat cells at ratio 1:5 (Target:Effector) after incubation with different concentrations of: A. Nimotuzumab. Cell viability was not significantly affected until treated with 1000 μg/ml of Nimotuzumab. B. Nmab-Fe₃O₄@Au nanoparticles, and C. Bis-Fe₃O₄@Au nanoparticles (nanoparticles conjugated to nimotuzumab and anti-CD3 scFv). The viability of co-cultured cells was significantly decreased following incubation with Bis-Fe₃O₄@Au NPs, compared with Nmab-Fe₃O₄@Au nanoparticle with ≥300 μg/ml iron. Data are reported as means±SD. 2000 μg/ml of Nimotuzumab versus Control ***p 0.0001, 300 μg/ml of Nmab-Fe₃O₄@Au NPs versus Control ***p 0.0003 and 700 μg/ml Nmab-Fe₃O₄@Au NPs versus Control **** p<0.0001, 300 and 700 g/ml of Bis-Fe₃O₄@Au NPs versus Control **** p<0.0001 (One-way ANOVA post hoc fisher LSD).

FIG. 9: Cytotoxicity assay of co-cultured A431 and Jurkat cells at ratio 1:5 (Target:Effector) after incubation with Nimotuzumab (1, left column), Nmab-Fe₃O₄@Au nanoparticles (2, middle column) and Bis-Fe₃O₄@Au nanoparticles (3, right column, bispecific NPs conjugated with anti-EGFR Nmab and αCD3scFv) at the same Nimotuzumab concentration. The viability of co-cultured cells was not significantly affected until treated with 1000 μg/ml of Nimotuzumab for cells treated with the antibody alone, while, the viability was significantly decreased following incubation with Bis-Fe₃O₄@Au nanoparticles, compared with Nmab-Fe₃O₄@Au NPs with a ≥43 μg/ml Nimotuzumab. Data are reported as means±SD.

FIG. 10: Cellular uptake of functionalized Fe₃O₄@Au NPs into cancer cell lines. A. Intracellular iron and gold content was quantitatively determined by ICP-MS after being incubated with 50 μg/ml iron for 12 hrs. B. Intracellular iron was detected by Prussian blue staining after the incubation with 50 μg/ml Fe₃O₄@Au NPs for 12 hrs (a: Fe₃O₄@Au NPs; b: Nmab-Fe₃O₄@Au NPs; c: Bis-Fe₃O₄@Au NPs, bispecific NPs conjugated with anti-EGFR Nmab and αCD3scFv). Uptake data is reported as mean+SD (×3).

FIG. 11: Cytotoxicity assay of co-cultures SkBr3 and Jurkat cells at ratio 1:5 (Target:Effector) after incubation with: A. Free Tmab B. Tmab-Fe₃O₄@Au NPs and C. Bis-Tmab-Fe₃O₄@Au-CD3 scFv NPs (bispecific NPs conjugated with anti-HER2 Tmab and αCD3scFv). The viability of co-cultured cells was significantly decreased following incubation with Bis-Tmab-Fe₃O₄@Au-CD3 scFv NPs, compared with Tmab-Fe₃O₄@Au NPs with a ≥300 μg/ml iron. Data are reported as means±SD. D: Cytotoxicity assay of co-cultures SkBr3 and Jurkat cells at ratio 1:5 (Target:Effector) after incubation with different concentrations of free Tmab, Tmab-Fe₃O₄@Au NPs (Tmab NPs), or Bis-Tmab-Fe₃O₄@Au-αCD3scFv NPs (Bis NPs; (bispecific NPs conjugated with anti-HER2 Tmab and αCD3scFv). The viability of co-cultured cells was significantly decreased following incubation with Bis-Tmab-Fe₃O₄@Au αCD3scFv NPs, compared with Tmab-Fe₃O₄@Au NPs, and compared with ≥43 μg/ml free Tmab. Free Tmab had no significant effect on cell viability below 200 μg/ml. Data are reported as means±SD.

FIG. 12: Characterization of Tmab-Fe₃O₄@Au NPs. A. Representative TEM image of flower shape NPs. B. Hydrodynamic diameter of NPs after conjugation with an average size of 149.3 nm.

FIG. 13: Cytokine release profile of EGFR-A431 targeting T cells. In a 96-well plate, 4×10³ cells/well cells were seeded then Jurkat E6-1 cells were added in 5:1 effector:target (E:T) ratio and incubated with anti-CD3 scFv, Bis-Fe₃O₄@Au NPs (bispecific NPs conjugated with anti-EGFR Nmab and αCD3scFv) and Nmab-Fe₃O₄@Au NPs for 24 hrs. Cytokine IL-2 secreted into the supernatant was determined by ELISA. The data is reported as mean±SD. Bis-NPs versus anti-CD3 scFv conjugated NPs, Nmab-NPs, and control unconjugated NPs *** p<0.0001 (One-way ANOVA post hoc fisher LSD).

FIG. 14. TEM representative image of dumbbell-like Fe₃O₄@Au nanoparticles. The diameter of the gold and iron nanoparticles are approximately 5 and 10 nm, respectively.

FIG. 15: Cytotoxicity assay performed in co-culture of A431 and Jurkat cells at ratio 1:5 (Target:Effector) with the following treatments at different concentrations of dumbbell-like NPs: A. Bare NPs, B. Bis-Fe₃O₄@Au NPs (bispecific NPs conjugated with anti-EGFR Nmab and αCD3scFv), and C. Nmab-Fe₃O₄@Au NPs. The viability of co-culture cells was significantly decreased following incubation with Bis-Fe₃O₄@Au NPs, compared with Nmab-Fe₃O₄@Au NPs with a ≥300 μg/ml iron.

FIG. 16: Characterization of spherical gold nanoparticles. A. TEM image of representative Au nanoparticles. B. UV-Visible absorption spectroscopy of the gold nanoparticles 521 nm.

FIG. 17: Cytotoxicity assay performed in co-culture of A431 and Jurkat cells at ratio 1:5 (Target:Effector) with the following treatments at different concentration of spherical AuNPs: A. Nmab-AuNPs, B. Bis AuNPs (bispecific NPs conjugated with anti-EGFR Nmab and αCD3scFv). The viability of co-culture cells was not significantly affected after incubation with Bis-AuNPs, compared with Nmab-AuNPs.

FIG. 18: Cytotoxicity assay performed in co-culture of MDA-MB 468 cells and Jurkat cells at ratio 1:5 (Target:Effector) with the following treatments at different concentrations: A. Free Nmab, B. Nmab-Fe₃O₄@Au NPs, and C. Bis-Nmab-Fe₃O₄@Au-αCD3scFv NPs (bispecific NPs conjugated with anti-EGFR Nmab and αCD3scFv). The viability of co-culture cells was significantly decreased following incubation with Bis-Nmab-Fe₃O₄@Au-αCD3scFv NPs, compared with Nmab-Fe₃O₄@Au NPs with a ≥300 μg/ml iron. Data are reported as means±SD. **** p<0.0001 (One-way ANOVA post hoc fisher LSD).

FIG. 19: Cytotoxicity assay of co-cultures MDA-MB 468 and Jurkat cells at ratio 1:5 (Target:Effector) after incubation with different concentrations of free Nmab, Nmab-Fe₃O₄@Au NPs (Nmab NPs), or Bis-Nmab-Fe₃O₄@Au-αCD3scFv (bispecific NPs conjugated with anti-EGFR Nmab and αCD3scFv). The viability of co-cultured cells was significantly decreased following incubation with Bis-Nmab-Fe₃O₄@Au-αCD3scFv NPs, compared with Nmab-Fe₃O₄@Au NPs, and compared with ≥43 μg/ml free Nmab. Free Nmab had no significant effect on cell viability below 100 μg/ml. Data are reported as means±SD.

FIG. 20: Cytotoxicity assay performed in co-culture of A431 and Jurkat cells at ratio 1:5 (Target:Effector) with the αCD3scFv-Fe₃O₄@Au-Nmab NPs at different concentrations. A. The viability of co-culture cells was significantly decreased following incubation with αCD3scFv-Fe₃O₄@Au-Nmab NPs, with 43 μg/ml Nmab. B. The viability of co-culture cells was significantly decreased following incubation with αCD3scFv-Fe₃O₄@Au-Nmab NPs, with a ≥300 μg/ml iron.

FIG. 21: Cytotoxicity assay of co-cultures MDA-MB 468 and Jurkat cells at ratio 1:5 (Target:Effector) after incubation with: A. Free Tmab B. Tmab-Fe₃O₄@Au NPs and C. Bis-Tmab-Fe₃O₄@Au-αCD3scFv NPs αCD3scFv (bispecific NPs conjugated with anti-HER2 Tmab and αCD3scFv). The viability of co-cultured cells was significantly decreased following incubation with Bis-Tmab-Fe₃O₄@Au-αCD3scFv NPs, compared with Tmab-Fe₃O₄@Au NPs with a ≥300 μg/ml iron. Data are reported as means±SD. * p<0.0001, ** p<0.0001, **** p<0.0001 (One-way ANOVA post hoc fisher LSD).

FIG. 22: Cytotoxicity assay of co-cultures MDA-MB 468 and Jurkat cells at ratio 1:5 (Target:Effector) after incubation with different concentrations of free Tmab, Tmab-Fe₃O₄@Au NPs (Tmab NPs), or Bis-Tmab-Fe₃O₄@Au-αCD3scFv NPs (Bis NPs; bispecific NPs conjugated with anti-HER2 Tmab and αCD3scFv). The viability of co-cultured cells was significantly decreased following incubation with Bis-Tmab-Fe₃O₄@Au-αCD3scFv NPs, compared with Tmab-Fe₃O₄@Au NPs, and compared with ≥43 μg/ml free Tmab. Free Tmab had no significant effect on cell viability below 200 μg/ml. Data are reported as means±SD.

FIG. 23: Cytotoxicity assay performed in co-culture of A431 and Jurkat cells at ratio 1:5 (Target:Effector) with bispecific anti-EGFR and anti-CD3 NPs, Panitumumab (Pmab)-Fe₃O₄@Au-αCD3scFv, at different concentrations. A. The viability of co-culture cells was significantly decreased following incubation with Pmab-Fe₃O₄@Au-αCD3scFv NPs with 10 μg/ml Pmab. B. The viability of co-culture cells was significantly decreased following incubation with Pmab-Fe₃O₄@Au-αCD3scFv NPs, with a ≥70 μg/ml iron.

FIG. 24: Characterization of silver iron nanoparticles (Fe₃O₄@Ag NPs). A. TEM image of representative silver iron nanoparticles. B. UV-Visible absorption spectroscopy of the silver iron nanoparticles 409 nm.

FIG. 25: Cytotoxicity assay performed in co-culture of A431 and Jurkat cells at ratio 1:5 (Target:Effector) with bispecific anti-EGFR and anti-CD3 NPs conjugated with a CPP, Nmab-Fe₃O₄@Au-αCD3scFv/CPP, at different concentrations. A. The viability of co-cultured A431 and Jurkat cells was significantly decreased following incubation with ≥70 μg/ml iron concentration of Nmab-Fe₃O₄@Au-αCD3scFv/CPP NPs. B. The viability of co-cultured A431 and Jurkat cells was significantly decreased following incubation with ≥10 μg/ml Nmab concentration of Nmab-Fe₃O₄@Au-αCD3scFv/CPP NPs.

FIG. 26: Cytotoxicity assay performed in co-culture of A431 and Jurkat cells at ratio 1:5 (Target:Effector) with the different concentrations of Nmab-Fe₃O₄@Au-αCD3scFv NPs, in the presence and absence of CPP. The viability of co-culture cells was significantly decreased following incubation with Nmab-Fe₃O₄@Au-αCD3scFv/CPP NPs (CPPBisNps, right columns) with a ≥35 μg/ml iron concentration, compared to Nmab-Fe₃O₄@Au-αCD3scFv NPs with conjugated CPP (Bis NPs, left columns).

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Currently available bispecific binders, especially those for engaging T cells and cancer cells (so-called bi-specific T cell engagers, or “BiTes”), suffer from low conformational and formulation stability and have a short serum half-life leading to their rapid elimination from the body. Several hundreds of different BiTe constructs have been reported so far, and most of these constructs suffer from such stability and half-life issues. This represents a significant limitation for the use of these antibodies for the treatment of cancer, where a longer serum half-life is essential for better therapeutic outcomes.

The present invention seeks to overcome one or more limitations of the bi-specific binders of the prior art. The present invention relates to nanoparticle-based bispecific binders (a nano-bioconjugate), comprising both an immune-cell engager and a cancer-specific engager, wherein the nano-bioconjugate is capable of interacting with both immune cells (such as T cells, NK cells and the like) and cancer cells.

This invention is thought to have various advantages over existing bispecifics including higher conformational and formulation stabilities. Further, it is believed that the nano-bioconjugates of the invention are likely to have a longer serum half-life because the size of the nanoparticles means that they would likely bypass renal filtration.

Advantageously, the nanoparticles of the present invention provide a 1^{st} and 2^{nd} binding protein (for binding cancer cells and immune cells, respectively), conjugated to different nanoparticle types. The presence of two different nanoparticle types (such as iron and gold), in a composite nanostructure, facilitates the easy attachment of different functional groups, enabling the conjugation of two different antibodies, or fragments thereof and thereby provide design flexibility. Moreover, the use of different nanoparticle types enables the use of different chemistries for conjugating the antigen binding proteins. This provides several advantages including the ability to control the ratios of each binding protein, and efficiencies in preparing the nanoparticle conjugates (eg fewer processing steps).

Moreover, the use of magnetic nanoparticles facilitates purification and preparation of the bioconjugates for therapeutic use.

### Definitions

Unless defined otherwise, technical and scientific terms used herein have the same meaning as commonly understood by one of ordinary skill in the art to which this invention belongs.

For the purposes of interpreting this specification, the following definitions will generally apply and whenever appropriate, terms used in the singular will also include the plural and vice versa.

The term “bispecific” means that the nanoparticle bioconjugate of the invention is able to specifically bind to at least two distinct antigenic determinants. Typically, a bispecific binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

As used herein, an “antigen” refers to a molecule or molecular structure that can be bound by a specific antibody or T-cell receptor. Antigens can be proteins, peptides (amino acid chains), polysaccharides (chains of monosaccharides/simple sugars), lipids, or nucleic acids.

In any aspect, the binding protein is any protein that can associate with or recognise or bind to a target moiety on the cancer or immune cell. Preferably, the target moiety is a protein endogenous to the target cell. The binding protein may comprise, consist essentially of or consist of an antigen binding domain. In the alternative, the binding protein may comprise, consist essentially of, or consist of a receptor or part thereof for binding the target moiety.

As used herein, a “target moiety” can be any moiety present on a target cell (eg a cancer or immune cell) that is capable of being bound by a binding protein. In any aspect, the target moiety (ie the target of the binding protein) is a protein that is presented, displayed or otherwise accessible on the cell surface or secreted by the cancer or immune cell. The target moiety may be constitutively presented, displayed or otherwise accessible on the cell surface, or only transiently presented, displayed or otherwise accessible on the cell surface. In one embodiment, the target is a protein that is transported and secreted from the cell such that it is not tethered to or part of the cell. The target may be constitutively secreted, or only transiently secreted.

The binding protein may comprise a receptor for a ligand or a binding partner for binding a target molecule. It will be appreciated that the binding partner may be in the form of any molecule that enables binding to the cancer or immune cell. For example, the binding partner may comprise or consist of a ligand, receptor, target or antigen binding domain. The binding partner may be a naturally occurring molecule that binds to the target. The binding partner may be non-naturally occurring or may be modified (e.g. by directed evolution) from a naturally occurring molecule. The binding partner may be naturally occurring but may be heterologous to the intracellular retention portion or linker.

In any aspect, the binding protein comprises, consists essentially of or consists of an antigen binding domain of an antibody or antigen binding fragment thereof. The antibody or antigen-binding fragment may have been modified or engineered, or is a human antibody. Examples of antibodies that have been modified or engineered are chimeric antibodies, humanized antibodies, multiparatopic antibodies (e.g., biparatopic antibodies), and multispecific antibodies (e.g., bispecific antibodies). Examples of antigen-binding fragments include Fab, Fab′, F(ab′)2, Fv, single chain antibodies (e.g., scFv), minibodies and diabodies.

In any aspect, the binding protein may bind to 2 different target moieties or 2 different epitopes on the same target moiety.

“Antibodies” or “immunoglobulins” or “Igs” are gamma globulin proteins that are found in blood, or other bodily fluids of verterbrates that function in the immune system to bind antigen, hence identifying and/or neutralising foreign objects. Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intrachain disulfide bridges.

H and L chains define specific Ig domains. More particularly, each H chain has at the N-terminus, a variable domain (V_{H}) followed by three constant domains (CH) for each of the α and γ chains and four CH domains for μ and ε isotypes. Each L chain has at the N-terminus, a variable domain (V_{L}) followed by a constant domain (CL) at its other end. The V_{L} is aligned with the V_{H} and the C_{L} is aligned with the first constant domain of the heavy chain (CHL).

Antibodies can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in ¾ sequence and function, e.g., humans express the following subclasses: IgG1, IgG2, IgG3, IgG4, IgA1, and IgA2. The L chain from any vertebrate species can be assigned to one of two clearly distinct types, called kappa and lambda, based on the amino acid sequences of their constant domains.

The constant domain includes the Fc portion that comprises the carboxy-terminal portions of both H chains held together by disulfides. The effector functions of antibodies such as ADCC are determined by sequences in the Fc region, which region is also the part recognised by Fc receptors (FcR) found on certain types of cells.

The pairing of a V_{H} and V_{L} together forms a “variable region” or “variable domain” including the amino-terminal domains of the heavy or light chain of the antibody. The variable domain of the heavy chain may be referred to as “V_{H}.” The variable domain of the light chain may be referred to as “V_{L}.” The V domain contains an “antigen binding site” that affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a β-sheet configuration and the hypervariable regions form loops connecting, and in some cases forming part of, the β-sheet structure.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196:901-917, 1987; Chothia et al., Nature 342:877-883, 1989; and/or Al-Lazikani et al., J. Mol. Biol. 273:927-948, 1997; the numbering system of Honnegher and Plükthun J. Mol. Biol. 309:657-670, 2001; or the IMGT system discussed in Giudicelli et al., Nucleic Acids Res. 25:206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9:133-139, 1995 established that the five C-terminal amino acids of heavy chain CDR2 are not generally involved in antigen binding

“Hypervariable region” refers to the regions of an antibody variable domain that are hypervariable in sequence and/or form structurally defined loops. Generally, antibodies comprise six hypervariable regions; three in the V_{H} (H1, H2, H3), and three in the V_{L} (L1, L2, L3).

“Framework” or “FR” residues are those variable domain residues other than the hypervariable region residues herein defined.

An “antigen binding site” generally refers to a molecule that includes at least the hypervariable and framework regions that are required for imparting antigen binding function to a V domain. An antigen binding site may be in the form of an antibody or an antibody fragment, (such as a mAb, single domain (SD)-mAb, dAb, Fab, SD-Fab, Fd, SD-Fv, Fv, F(ab′)2 or scFv) in a method described herein. As used herein, the termins antigen binding site, antigen binding moiety, and antigen binding domain may be used interchangeably. It will be appreciated that an antigen binding protein may comprise or consist of an antigen binding domain.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

The terms “binds to”, “specifically binds to” or “specific for” with respect to a targeting moiety, refers to an binding domain that recognises and binds to a specific ligand or antigen, does not substantially recognise or bind to other molecules in a sample. In the context of antigen binding proteins, the antigen binding domain or targeting moiety that binds specifically to an antigen from one species also may bind to that antigen from another species. This cross-species reactivity is typical of many antibodies and therefore not contrary to the definition that the antigen-binding domain is specific. An antigen-binding domain that specifically binds to an antigen may bind also to different allelic forms of the antigen (allelic variants, splice variants, isoforms etc.) or homologous variants of this antigen from the same gene family. This cross reactivity is typical of many antibodies and therefore not contrary to the definition that the antigen-binding domain is specific.

An “intact” or “whole” antibody is one that comprises an antigen-binding site as well as a C_{L} and at least heavy chain constant domains, CH1, CH2 and CH3. The constant domains may be native sequence constant domains (e.g. human native sequence constant domains) or amino acid sequence variant thereof.

“Whole antibody fragments including a variable domain” include SD-mAb, Fab, Fab′, F(ab′)2, and Fv fragments; diabodies; linear antibodies, single-chain antibody molecules; and multi-specific antibodies formed from antibody fragments.

The “Fab fragment” consists of an entire L chain along with the variable region domain of the H chain (V_{H}), and the first constant domain of one heavy chain (CHI). Each Fab fragment is monovalent with respect to antigen binding, i.e., it has a single antigen-binding site.

A “Fab′ fragment” differs from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group.

A “F(ab′)2 fragment” roughly corresponds to two disulphide linked Fab fragments having divalent antigen-binding activity and is still capable of cross-linking antigen.

An “Fv” is the minimum antibody fragment that contains a complete antigen-recognition and binding site. This fragment consists of a dimer of one heavy and one light chain variable region domain in tight, non-covalent association.

In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody.

“Single-chain Fv” also abbreviated as “sFv” or “scFv” are antibody fragments that comprise the V_{H} and V_{L} antibody domains connected to form a single polypeptide chain. Preferably, the scFv polypeptide further comprises a polypeptide linker between the V_{H} and V_{L} domains which enables the scFv to form the desired structure for antigen binding.

A “single variable domain” is half of an Fv (comprising only three CDRs specific for an antigen) that has the ability to recognise and bind antigen, although generally at a lower affinity than the entire binding site.

“Diabodies” refers to antibody fragments with two antigen-binding sites, which fragments comprise a heavy-chain variable domain (V_{H}) connected to a light-chain variable domain (V_{L}) in the same polypeptide chain (V_{H}-V_{L}). The small antibody fragments are prepared by constructing sFv fragments (see preceding paragraph) with short linkers (about 5-10 residues) between the V_{H} and V_{L} domains such that interchain but not intrachain pairing of the V domains is achieved, resulting in a bivalent fragment, i.e., fragment having two antigen-binding sites.

Diabodies may be bivalent or bispecific. Bispecific diabodies are heterodimers of two “crossover” sFv fragments in which the V_{H} and V_{L} domains of the two antibodies are present on different polypeptide chains. Triabodies and tetrabodies are also generally known in the art.

In any embodiment, the binding protein may be derived from a human antibody or may be a humanised form of a non-human antibody.

A “human antibody” refers to an antibody that possesses an amino acid sequence that corresponds to that of an antibody produced by a human. Human antibodies can be produced using various techniques known in the art, including phage-display libraries. Human antibodies can be prepared by administering the antigen to a transgenic animal that has been modified to produce such antibodies in response to antigenic challenge, but whose endogenous loci have been disabled.

“Humanised” forms of non-human (e.g., rodent) antibodies are chimeric antibodies that contain minimal sequence derived from the non-human antibody. For the most part, humanised antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanised antibodies may comprise residues that are not found in the recipient antibody or in the donor antibody. These modifications are made to further refine antibody performance. In general, the humanised antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanised antibody optionally also will comprise at least a portion of an immunoglobulin constant region (Fc), typically that of a human immunoglobulin.

“Monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site or determinant on the antigen. In addition to their specificity, the monoclonal antibodies are advantageous in that they may be synthesised uncontaminated by other antibodies. Monoclonal antibodies may be prepared by the hybridoma methodology. The “monoclonal antibodies” may also be isolated from phage antibody libraries using molecular engineering techniques.

As used herein, the term “antigen” is intended to include substances that bind to or evoke the production of one or more antibodies and may comprise, but is not limited to, proteins, peptides, polypeptides, oligopeptides, lipids, carbohydrates, and combinations thereof, for example a glycosylated protein or a glycolipid. The term “antigen” as used herein refers to a molecular entity that may be expressed on a target cell and that can be recognised by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

“Epitope” generally refers to that part of an antigen that is bound by the antigen binding site of an antibody. An epitope may be “linear” in the sense that the hypervariable loops of the antibody CDRs that form the antigen binding site bind to a sequence of amino acids as in a primary protein structure. In certain embodiments, the epitope is a “conformational epitope” i.e. one in which the hypervariable loops of the CDRs bind to residues as they are presented in the tertiary or quaternary protein structure.

The terms “immune cell” or “immune effector cell” refer to a cell that may be part of the immune system and executes a particular effector function such as alpha-beta T cells, NK cells, NKT cells, B cells, Breg cells, Treg cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes or macrophages or any hematopoietic progenitor cells such as pluripotent stem cells and early progenitor subsets that may mature or differentiate into somatic cells.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, the term “subject” refers to a mammal such as mouse, rat, cow, pig, goat, chicken, dog, monkey or human. Preferentially, the subject is a human. The subject may be a subject suffering from a disorder such as cancer (a patient), but the subject also may be a healthy subject. As used herein, the terms “subject”, “individual” and “patient” may be used interchangeable.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

The term “treat” (treatment of) a disorder as used herein means to reduce the frequency or severity of at least one sign or symptom of a disease or disorder experienced by a subject.

The term “prevent” as used herein, is intended to refer to at least the reduction of likelihood of the risk of (or susceptibility to) acquiring a disease or disorder (i.e., causing at least one of the clinical symptoms of the disease not to develop in a individual that may be exposed to or predisposed to the disease but does not yet experience or display symptoms of the disease). Biological and physiological parameters for identifying such patients are provided herein and are also well known by physicians. For example, prevention of an aberrant immune response, may be characterised by an absence of an increased release of cytokines following treatment with a cellular immunotherapeutic agent.

### Binding Proteins

The nanoparticle bioconjugates of the invention comprise a 1^{st} nanoparticle to which is conjugated a 1^{st} binding protein for binding a target moiety of a cancer cell, and a 2^{nd} nanoparticle, to which is conjugated a 2^{nd} binding protein, for binding a target moiety of an immune cell, preferably an antigen of a T cell.

The 1^{st} and 2^{nd} binding proteins (or binding moieties) of the bioconjugates of the invention may be in the form of any antigen binding protein described herein, including an antibody (including a human or humanised antibody), variant or fragment thereof.

In any aspect, the binding proteins comprise or consists of an antigen binding domain of an antibody or antigen binding fragment thereof. The antibody or antigen-binding fragment may have been modified or engineered, or is a human antibody. Examples of antibodies that have been modified or engineered are chimeric antibodies, humanized antibodies, multiparatopic antibodies (e.g., biparatopic antibodies), and multispecific antibodies (e.g., bispecific antibodies). Examples of antigen-binding fragments include Fab, Fab′, F(ab′)₂, Fv, single chain antibodies (e.g., scFv), minibodies and diabodies.

In any aspect, the antigen binding protein may be in the form of:
(i) a single chain Fv fragment (scFv);
(ii) a dimeric scFv (di-scFv); or
(iii) one of (i) or (ii) linked to a constant region of an antibody, Fc or a heavy chain constant domain (CH) 2 and/or CH3.

Further, as described herein, the antigen binding protein may be in the form of:
(i) a diabody;
(ii) a triabody;
(iii) a tetrabody;
(iv) a nanobody;
(v) an ibody;
(vi) a V_{H} domain
(vii) a Fab;
(viii) a F(ab′)2;
(ix) a Fv; or
(x) one of (i) to (ix) linked to a constant region of an antibody, Fc or a heavy chain constant domain (CH) 2 and/or CH3.

The foregoing antigen binding proteins can also be referred to as antigen binding domains of antibodies.

In certain examples, the 1^{st} and 2^{nd} antigen binding proteins are of the same architecture, eg: both proteins may be in the form of an scFv, or other similar antibody fragment. Alternatively, both proteins may be in the form of an IgG, including an IgG of any class.

In alternative examples, the 1^{st} and 2^{nd} antigen binding proteins are of differing architectures. For example, the 1^{st} antigen binding protein (ie for binding a cancer cell antigen) is preferably in the form of an IgG while the 2^{nd} antigen binding protein (ie for binding an antigen on an immune cell, preferably a T cell), may be in the form of an antibody fragment, such as an scFv.

The T cell antigen binding protein preferably comprises at least one binding molecule capable of binding to an activating T cell antigen. In a particular embodiment, the T cell binding protein comprises not more than one antigen binding moiety capable of specific binding to an activating T cell antigen. Accordingly, in one embodiment the T cell antigen binding protein provides monovalent binding to the activating T cell antigen.

In a particular embodiment the T cell antigen is CD3, particularly human or cynomolgus CD3, most particularly human CD3. In some embodiments, the T cell antigen is the epsilon subunit of CD3.

Antibodies for binding to CD3 are known in the art, as are antibodies for binding CD3episilon. In certain embodiments, the binding protein comprises part or all of the antigen binding domain of a known CD3-binding protein, optionally selected from H2C, OKT3, UCGT1 and FN18.

In one embodiment the T cell antigen binding scFv can compete with monoclonal antibody H2C (described in PCT publication no. WO2008/119567) for binding an epitope of CD3. In another embodiment, the T cell antigen binding scFv can compete with monoclonal antibody V9 (described in Rodrigues et al, Int J Cancer Suppl 7, 45-50 (1992) and U.S. Pat. No. 6,054,297) for binding an epitope of CD3. In yet another embodiment, the T cell antigen binding scFv can compete with monoclonal antibody FN18 (described in Nooij et al, Eur J Immunol 19, 981-984 (1986)) for binding an epitope of CD3.

In one embodiment, the T cell binding protein is specific for CD3 and comprises the three CDRs of a heavy chain variable region sequence that is at least about 80%, 85%, 90%, 95%, 96%, 97%, 98%, 99% or 100% identical to SEQ ID NO: 1 and/or the three CDRs of a light chain variable region sequence that is at least about 80%, 85%, 90%, 95%, 96%, 97%, 98%, 99% or 100% identical to SEQ ID NO: 3, or variants thereof that retain functionality.

In any embodiment, the T cell binding protein comprises a heavy chain variable region sequence that is at least about 80%, 85%, 90%, 95%, 96%, 97%, 98%, 99% or 100% identical to SEQ ID NO: 1 and a light chain variable region sequence that is at least about 80%, 85%, 90%, 95%, 96%, 97%, 98%, 99% or 100% identical to SEQ ID NO: 3, or variants thereof that retain functionality, including a humanised variant thereof. In one embodiment, the anti-CD3 binding protein comprises the sequence of the OKT3 antibody fragment with code 1SY6 obtained from Protein Data Bank (https://www.rcsb.org/structure/1sy6).

In another particular embodiment the T cell antigen is CD16, particularly human or cynomolgus CD16, most particularly human CD16. Antibodies for binding to CD16 are known in the art.

In one embodiment, the T cell binding protein is specific for CD16 and comprises the three CDRs of a heavy chain variable region sequence that is at least about 80%, 85%, 90%, 95%, 96%, 97%, 98%, 99% or 100% identical to SEQ ID NO: 10 and/or the three CDRs of a light chain variable region sequence that is at least about 80%, 85%, 90%, 95%, 96%, 97%, 98%, 99% or 100% identical to SEQ ID NO: 11, or variants thereof that retain functionality.

In any embodiment, the T cell binding protein comprises a heavy chain variable region sequence that is at least about 80%, 85%, 90%, 95%, 96%, 97%, 98%, 99% or 100% identical to SEQ ID NO: 10 and a light chain variable region sequence that is at least about 80%, 85%, 90%, 95%, 96%, 97%, 98%, 99% or 100% identical to SEQ ID NO: 11, or variants thereof that retain functionality, including a humanised variant thereof. In one embodiment, the anti-CD16 binding protein comprises the sequence of the anti-CD16 antibody fragment with catalogue number #1151-V obtained from ViroGen).

In alternative embodiments, the T cell antigen is selected from the group consisting of: CD2, CD4, CD7, CD8, PD1, CTLA4, KIR, CD16, CD94, CD161, NTBA, CD19, CD20, CD22, CD30, CD33, CD38, CD40L, CD44, CD56, CD79b, CD80, CD86, CD135, CD137, CD138, CD154, EphA2, EGFR, or any combination thereof. It will be appreciated that this is not an exhaustive list of T cell antigens and it is within the purview of the skilled person to be able to identify a suitable T cell antigen, and corresponding antigen binding protein for binding to same.

Antibodies for binding such T cell antigens are also known in the art. For example, antibodies for binding CD38 include Daratumumab, felzartamab, isatuximab, mezagitamab; antibodies for binding CD7 include Grisnilimab; antibodies for binding PD1 include balstilimab, budigalimab, camrelizumab, cemiplimab, cetrelimab, dostarlimab, ezabenlimab, geptanolimab, lodapolimab, izuralimab; antibodies for binding CTLA-4 include ipilimumab, nurulimab, pavunalimab, quavonlimab, tremelimumab, vudalimab, zalifrelimab; antibdoies for binding; antibodies for binding CD30 include Iratumumab, SGN-30, 5F11. The skilled person can make use of the antigen binding domains from any known antibody for producing a nano-bioconjugate according to the present invention.

The cancer cell antigen binding protein will be understood to be any protein capable of specifically binding to an antigen on a cancer cell, such as a tumour associated antigen or a tumour-specific antigen.

As used herein “tumour-associated antigen” or a cancer cell antigen refers to an antigen that is expressed by cancer cells (the term “tumour-antigen” may also be used to refer to same). Tumour antigens are proteins that are produced by tumour cells that elicit an immune response, particularly T-cell mediated immune responses. Tumour antigens are well known in the art and include, for example, a glioma-associated antigen, carcinoembryonic antigen (CEA), β-human chorionic gonadotropin, alpha fetoprotein (AFP), lectin-reactive AFP, thyroglobulin RAGE-1, MN-CAIX, human telomerase reverse transcriptase, RU1, RU2 (AS), intestinal carboxyl esterase, mut hsp70-2, M-CSF, hK4 prostase, prostate-specific antigen (PSA), PAP, NY-ESO-1, LAGE-1a, p53, P501S prostein, PSMA, Her2/neu, survivin and telomerase, prostate-carcinoma tumour antigen-1 (PCTA-1), MAGE, ELF2M, neutrophil elastase, ephrinB2, CD22, insulin growth factor (IGF)-I, IGF-II, IGF-I receptor and mesothelin.

The cancer cell antigen binding protein is preferably one capable of specific binding to any one of the following cancer cell antigens: endothelial growth factor receptor (EGFR), human epidermal growth factor receptor 2 (HER2), Fibroblast Activation Protein (FAP), Melanoma-associated Chondroitin Sulfate Proteoglycan (MCSP), Carcinoembryonic Antigen (CEA), carbonic anhydrase IX (CAIX), prostate specific antigen (PSMA), CD33, CD19, CD20, mesothelin, GPC3, MUC1, GD2, CEA, EpCAM, LeY, PCSA, Clec9a, CD276, PD-L1 and PD-L2 or any other cancer antigen described herein.

In one embodiment, the tumour antigen comprises one or more antigenic cancer epitopes associated with a malignant tumour. Malignant tumours express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-foetal antigens such as carcinoembryonic antigen (CEA). In B-cell lymphoma the tumour-specific idiotype immunoglobulin constitutes a truly tumour-specific immunoglobulin antigen that is unique to the individual tumour. B-cell differentiation antigens such as CD 19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

The type of tumour antigen referred to in the invention may also be a tumour-specific antigen (TSA). A TSA is unique to tumour cells and does not occur on other cells in the body. A tumour-associated antigen (TAA) is not unique to a tumour cell and instead is also expressed on a normal cell under conditions that fail to induce a state of immunologic tolerance to the antigen. The expression of the antigen on the tumour may occur under conditions that enable the immune system to respond to the antigen. TAAs may be antigens that are expressed on normal cells during foetal development when the immune system is immature and unable to respond or they may be antigens that are normally present at extremely low levels on normal cells but which are expressed at much higher levels on tumour cells. Those tumour-associated antigens of greatest clinical interest are differentially expressed compared to the corresponding non-tumour tissue and allow for a preferential recognition of tumour cells by specific T-cells or immunoglobulins.

Non-limiting examples of TSA or TAA antigens include the following: Differentiation antigens such as MART-1/MelanA (MART-1), gp 100 (Pmel 17), tyrosinase, TRP-1, TRP-2 and tumour-specific multilineage antigens such as MAGE-1, MAGE-3, BAGE, GAGE-1, GAGE-2, p15; overexpressed embryonic antigens such as CEA; overexpressed oncogenes and mutated tumour-suppressor genes such as p53, Ras, HER-2/neu; unique tumour antigens resulting from chromosomal translocations; such as BCR-ABL, E2A-PRL, H4-RET, 1GH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p 180erbB-3, c-met, nm-23H 1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA195, CA242, CA-50, CAM43, CD68\P 1, CO-029, FGF-5, G250, Ga733\EpCAM, HTgp-175, M344, MA-50, MG7-Ag, MOV 18, NB/70K, NY-CO-1, RCAS 1, SDCCAG16, TA-90Wac-2 binding protein\cyclophilin C-associated protein, TAAL6, TAG72, TLP, and TPS. Particularly preferred examples of target cell antigens in accordance with the present invention include: CD33 (Siglec-3), CD123 (IL3RA), CD135 (FLT-3), CD44 (HCAM), CD44V6, CD47, CD184 (CXCR4), CLEC12A (CLL1), LeY, FRp, MICA/B, CD305 (LAIR-1), CD366 (TIM-3), CD96 (TACTILE), CD133, CD56, CD29 (ITGB1), CD44 (HCAM), CD47 (IAP), CD66 (CEA), CD112 (Nectin2), CD117 (c-Kit), CD133, CD146 (MCAM), CD155 (PVR), CD171 (LI CAM), CD221 (IGF1), CD227 (MUC1), CD243 (MRD1), CD246 (ALK), CD271 (LNGFR), CD19, CD20, GD2, and especially EGFR, mesothelin, GPC3, MUC1, HER2, GD2, CEA, EpCAM, LeY, PCSA and CD276.

The skilled person will be familiar with antigen binding domains described in the literature, for binding to any one or more of the above listed cancer antigens. Certain non-limiting examples follow although it will be appreciated that the scope of the present invention should not be limited to the specific antigen binding proteins described herein. Moreover, once the skilled person has determined which cancer cell requires treating, the skilled person can then readily determine a suitable antigen present on the cancer cell for targeting, with reference to the known literature regarding the cancer cell type. The skilled person can then make use of any known or new antigen binding domains for binding to the cancer antigen, for conjugating to a nanoparticle as described herein in order to generate a suitable bioconjugate for treatment of the cancer. Capacity of the bioconjugate for binding to the target antigen can be confirmed using common laboratory techniques, including those referenced herein in the examples.

In one embodiment the cancer cell antigen binding protein is capable of specifically binding to Epidermal Growth Factor Receptor (EGFR). In another embodiment the binding protein comprises a scFv molecule that can compete with monoclonal antibody GA201 for binding to an epitope of EGFR (see PCT publication WO 2006/082515, incorporated herein by reference in its entirety).

In one embodiment, the binding protein that is specific for EGFR comprises the three CDRs of a heavy chain variable region sequence that is at least about 80%, 85%, 90%, 95%, 96%, 97%, 98%, 99% or 100% identical to SEQ ID NO: 6; and/or the three CDRs of a light chain variable region sequence that is at least about 80%, 85%, 90%, 95%, 96%, 97%, 98%, 99% or 100% identical to SEQ ID NO: 7, or variants thereof that retain functionality.

In certain embodiments, the binding protein specific for EGFR comprises a heavy chain variable region sequence that is at least about 80%, 85%, 90%, 95%, 96%, 97%, 98%, 99% or 100% identical to SEQ ID NO: 6 and a light chain variable region sequence that is at least about 80%, 85%, 90%, 95%, 96%, 97%, 98%, 99% or 100% identical to SEQ ID NO: 7, or variants thereof that retain functionality. In one embodiment, the binding protein that is specific for EGFR comprises the Nimotuzumab antibody (Nmab) with the code 3GKW obtained from the Protein Data Bank (https://www.rcsb.org/structure/3gkw).

In one embodiment, the binding protein that is specific for EGFR comprises the three CDRs of a heavy chain variable region sequence that is at least about 80%, 85%, 90%, 95%, 96%, 97%, 98%, 99% or 100% identical to SEQ ID NO: 9; and/or the three CDRs of a light chain variable region sequence that is at least about 80%, 85%, 90%, 95%, 96%, 97%, 98%, 99% or 100% identical to SEQ ID NO: 8, or variants thereof that retain functionality.

In certain embodiments, the binding protein specific for EGFR comprises a heavy chain variable region sequence that is at least about 80%, 85%, 90%, 95%, 96%, 97%, 98%, 99% or 100% identical to SEQ ID NO: 9 and a light chain variable region sequence that is at least about 80%, 85%, 90%, 95%, 96%, 97%, 98%, 99% or 100% identical to SEQ ID NO: 8, or variants thereof that retain functionality. In one embodiment, the binding protein that is specific for EGFR comprises the Panitumumab antibody (Pmab) with the code 5SX4 obtained from the Protein Data Bank (https://www.rcsb.org/structure/5sx4).

In one embodiment the cancer cell antigen binding protein is capable of specifically binding to HER2. In a one embodiment, the binding protein that is specific for HER2 comprises the three CDRs of a heavy chain variable region sequence that is at least about 80%, 85%, 90%, 95%, 96%, 97%, 98%, 99% or 100% identical to SEQ ID NO: 4 and/or the three CDRs of a light chain variable region sequence that is at least about 80%, 85%, 90%, 95%, 96%, 97%, 98%, 99% or 100% identical to SEQ ID NO: 5, or variants thereof that retain functionality. In any embodiment, the binding protein comprises a heavy chain variable region sequence that is at least about 80%, 85%, 90%, 95%, 96%, 97%, 98%, 99% or 100% identical to SEQ ID NO: 4 and a light chain variable region sequence that is at least about 80%, 85%, 90%, 95%, 96%, 97%, 98%, 99% or 100% identical to SEQ ID NO: 5, or variants thereof that retain functionality. In one embodiment, the binding protein that is specific for HER2 comprises the Trastuzumab antibody (Tmab) with the code DB00072 obtained from the online Drug Bank (https://go.drugbank.com/drugs/DB00072).

### Magnetic and Metallic Nanoparticles

The skilled person will appreciate that various nanoparticles may be used in order to generate the bioconjugates of the invention. Typically nanoparticles with superparamagnetic, crystallinity, physically and chemically stability, environmentally safety, and biocompatibility are preferred.

In any embodiment of the invention, the magnetic nanoparticle may be selected from: an Fe₃O₄ (magnetite), an FeCl₂, an Fe(Co)₅ (iron pentacarbonyl), maghemite (y-Fe₂O₃) or a hematite (a-Fe₂O₃) nanoparticle. In preferred embodiments, the magnetic nanoparticle is Fe₃O₄. In some embodiments, the mean diameter of the magnetic nanoparticle is about 14 nm.

In any embodiment, the metallic nanoparticle may be selected from: a gold (Au), a silver (Ag), platinum (Pt) or palladium (Pd), copper (Cu), nickel (Ni), cobalt (Co), or an alloy of two or more thereof or nanoparticle.

Preferably, the metallic nanoparticle is a gold or silver nanoparticle. In particularly preferred embodiments, the metallic nanoparticle is a gold nanoparticle.

In any embodiment, the nanoparticle bioconjugate is comprised of iron and gold nanoparticles, preferably wherein the bioconjugate comprises a central gold nanoparticle surrounded by 3-4 or more iron nanoparticles to form a flower-shaped nanoparticle.

In any embodiment, the nanoparticle bioconjugates of the invention may be between about 5 nm to about 200 nm. In any embodiment, the nanoparticle bioconjugates of the invention are between about 35 nm to 80 nm, preferably about 40 nm.

In any embodiment, the nanoparticle bioconjugates of the invention may have a polymer coating to increase their dispersibility and/or stability. Examples of polymers for nanoparticle surface modification include PEG, polydopamine, dextram, PVA, PAA, starch, or chitosam. In preferred embodiments, the nanoparticle bioconjugates of the invention have a PEG coating.

### Conjugation of Antigen Binding Domains to Nanoparticles

The skilled person will be familiar with standard techniques for conjugating antigen binding proteins to nanoparticles, including to a metallic or magnetic nanoparticle as described herein.

For example, standard techniques, such as those described herein in the Examples, may be used for covalently attaching antigen binding proteins to nanoparticles. Further examples of methods for the conjugation of nanoparticles to antigen binding proteins include those described in the art (see for example, Jazayeri et al. (2016) Various methods of gold nanoparticles (GNPs) conjugation to antibodies. Sensing and Bio-Sensing Research 9:17-22; and Cruz and Kayser (2019) Synthesis and Enhanced Cellular Uptake In Vitro of Anti-HER2 Multifunctional Gold Nanoparticles. Cancer 11 (870): 1-22; incorporated herein by reference).

The skilled person will appreciate that conjugation methods will depend on the functional groups that are available on the nanoparticle coating, linkers used, and the ligands to be attached to the nanoparticle. Examples of suitable conjugation methods include: carboxyl linkage (including to carboxyl groups present on the target binding protein using carbodiimide), maleimide linkage, azide linkage, biotin linkage, hydroxyl linkage, methyl linkage, neutravidin linkage, NHS (N-hydroxy succinimide) moiety linkage, thiol linkage, PEG moiety linkage, amide linkage, hydrazone linkage, amine linkage (including to amino groups present on the target binding protein using carbodiimide).

In preferred embodiments, the conjugation method is covalent conjugation involving amide linkage, hydrazone linkage and/or amine linkage; achieved by carbodiimide and/or malemide chemistry.

In some embodiments, conjugation is achieved using commercially available nanoparticle conjugation kits (e.g. supplied by Ocean NanoTech) such as carboxyl magnetic iron oxide nanoparticle kits, or amine iron oxide nanoparticle kits, or modified protocols based on the methods described in such kits. Again, these techniques will be within the skill set of the skilled person.

In any embodiment, the antigen binding proteins may be conjugated to a nanoparticle using a method as described herein in the Examples.

In preferred embodiments, the 1^{st} and 2^{nd} binding proteins are covalently conjugated to the nanoparticles via different chemistries. Utilisation of differing chemistries facilitates preparation of the composite nanoparticles.

In one example, one binding protein may be covalently conjugated to a magnetic nanoparticle via a polyethylene glycol (PEG) moiety and the other binding protein may be covalently conjugated to a metallic nanoparticle via a thiol linker, wherein optionally said thiol is at least one of thioctic acid, monothioctic acid, dithioctic acid, and trithioctic acid.

### Conditions to be Treated, Dosage and Administration

The bispecific nanoparticles of the invention have particularly utility for the treatment or prevention of cancers.

Broad examples include breast tumors, colorectal tumors, adenocarcinomas, mesothelioma, bladder tumors, prostate tumors, germ cell tumor, hepatoma/cholongio, carcinoma, neuroendocrine tumors, pituitary neoplasm, small round cell tumor, squamous cell cancer, melanoma, atypical fibroxanthoma, seminomas, nonseminomas, stromal leydig cell tumors, Sertoli cell tumors, skin tumors, kidney tumors, testicular tumors, brain tumors, ovarian tumors, stomach tumors, oral tumors, bladder tumors, bone tumors, cervical tumors, esophageal tumors, laryngeal tumors, liver tumors, lung tumors, vaginal tumors and Wilm's tumor.

Examples of particular cancers include but are not limited to adenocarcinoma, adenoma, adenofibroma, adenolymphoma, adontoma, AIDS related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, branchioma, CNS tumours, carcinoid tumours, cervical cancer, childhood brain tumours, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukaemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukaemia, leukosarcoma, leydig cell tumour, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's-macroglobulinemia and Wilms' tumour.

In some examples, a nanoparticle bioconjugate as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing a nanoparticle bioconjugate into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding site dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding site of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, a nanoparticle bioconjugate of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding site of the present invention.

Suitable dosages of a nanoparticle bioconjugate of the present invention will vary depending on the specific nanoparticle bioconjugate, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED₅₀ of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC₅₀ (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a nanoparticle bioconjugate described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding site(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding site(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding site(s), rather the present invention encompasses any amount of the antigen binding site(s) sufficient to achieve the stated result in a subject.

The present invention additionally comprises a kit comprising a nanoparticle bioconjugate of the invention. The kit can additionally comprise a pharmaceutically acceptable carrier.

Optionally a kit of the invention is packaged with instructions for use in a method described herein according to any example.

In preferred embodiments, the kit comprises components to generate a nanoparticle bioconjugate of the invention, for example, the components of the nanoparticles and the binding proteins for conjugating thereto.

### EXAMPLES

Here, the inventors report the development of a flower-like nanoparticle platform for combination delivery of two antigen-binding proteins. These nanoparticles, termed as bispecific nanoflowers comprise binding domains for binding an antigen on tumor cells (eg for binding EGFR using the antibody Nimotuzumab or Panitumumab), while simultaneously causing T-cell activation via binding to T cells (eg by using an anti-CD3 scFv or anti-CD16 scFv), also conjugated to the nanoparticle. By chemically conjugating the antibodies in a nanoparticle platform, the nanoflowers of the invention are expected to result in a synergy arising from the two binding proteins which is examined in vitro in cancer cells.

The inventors believe that this is the first proof-of concept demonstrating the synergistic effect of a dual antibody targeted immunotherapy in cancer via iron oxide-gold nanoparticles. In one example, the developed nano-delivery system shows that an exemplary bispecific iron oxide-gold nanoparticle is an effective bispecific bioconjugate for retargeting T cell toward EGFR-expressing cells in vitro, with a 69.7% higher rate than that of the free Nimotuzumab.

### Example 1: Materials and Methods

### Materials

Hydrogen tetrachloroaurate (III) trihydrate (HAUCL4·3H2O), iron pentacarbonyl (Fe (CO)₅), oleylamine, oleic acid, 1-octadecene, α,ω-Bis {2-[(3-carboxy-1-oxopropyl)amino]ethyl}PEG (Mr=3,000), N-hydroxy succinimide (NHS), N-(3-dimethylaminopropyl)NO-ethylcarbodiimide hydrochloride (EDC), 4-aminophenyl β-D-glucopyranoside, (2-(N-morpholino) ethane sulfonicacid) (MES), PD-10 desalting columns were purchased from Sigma-Aldrich (Sydney, NSW, AUS). 1,2 Hexadecandiol was purchased from TLC chemicals. MTT (3-[4,5-dimethylthiazol-2-yl]-2,5-diphenyltetrazoliumbromide), penicillin and streptomycin solutions were purchased from Invitrogen. All chemicals employed in the syntheses were used without further purification. Nimotuzumab (145 kDa) monoclonal antibody was from Biocon, (Bangalore, India) and is also referred to herein as Nmab. Trastuzumab (also referred to herein as Tmab) was from Genentech. Panitumumab (referred to herein as Pmab) was from Amgen. Single chain variable fragments for anti-CD3 scFv (28-30 KDa) were synthesized by the inventors as well as purchased from Virogen (Brighton, MA, USA). Single chain variable fragments for anti-CD16 scFv were purchased from Virogen (Brighton, MA, USA).

### Instrumentation and Characterization of NPs

Nanoparticles (NPs) were visualized by transmission electron microscopy (TEM), using a JEOL microscope (JEOL JEM 1400, Gatan Inc., USA), operating at acceleration voltage 120 kV. 8 μl of diluted (dilution ratio 1:3) NPs was applied onto a carbon coated copper grid. The grid was left at room temperature for 2 hours to dry and imaged using the transmission electron microscope.

A UV-Visible spectrophotometer (UV-Vis, Shimadzu, Japan) was used for the absorption spectra of Fe₃O₄@Au NPs, Fe₃O₄@AgNPs and antibody conjugation in hexane solution or aqueous solution, depending on the nature of the NPs. The hydrodynamic size distribution and zeta potential of NPs were measured in triplicate by a particle size analyzer Zeta-sizer Nano ZS90 (Malvern, Worcestershire, UK) at a scattering angel of 90 at 25° C., adjusted for reflective index of the dispersant. It reports the particle size distribution, mean particle diameter and the polydispersity index. Fourier transform infrared spectroscopy (FTIR) was recorded on a Shimadzu IRT racer-100 spectrophotometer. Inductively coupled plasma mass spectrometry using a PerkinElmer Nexion 300×ICP-MS instrument (PerkinElmer, Waltham, MA, USA) was used to determine the final concentration of NPs. For this, the NPs were atomized with aqua regia. All mass spectra were acquired using a Matrix-assisted laser desorption ionization/time-of-flight (MALDI-TOF) Bruker Autoflex Speed LRF, with a 1000 Hz Smartbeam-II laser. The spectra were recorded in positive reflection mode using an accelerating voltage of 9 kV, Linear positive-ion mode, Ion Source 1 19.5 kV, Ion Source 2:16.5 kV, Matrix Suppression Deflection on and suppress up to 18000 Da, Mass Range: 20,000-400,000 Da. Data analysis was performed using flex Control version 3.4, flex Analysis version 3.4.

SDS-PAGE was used to evaluate the NPs antibodies conjugation, samples were loaded on SDS-PAGE and electrophoresis was carried out at 100V for 30 min followed by incubation with Coomassie brilliant blue to detect the protein bands. Visualization and analysis of proteins band were carried out with the Bio-Rad Gel Doc™ XR system and analysed using Bio-Rad Image software. Nano-Drop 1000 (Thermo-Scientific) spectrophotometer measurements were used to confirm the conjugation, measurements were taken by measuring absorbance at 280 nm with a 0.1×PBS baseline. Enzyme linked immune assay (ELISA) was conducted, absorbance measurements were taken at 450 nm through a microplate reader. “Naked” NPs and conjugated NPs were measured in the presence of positive control (Herceptin®) and negative control (Assay buffer).

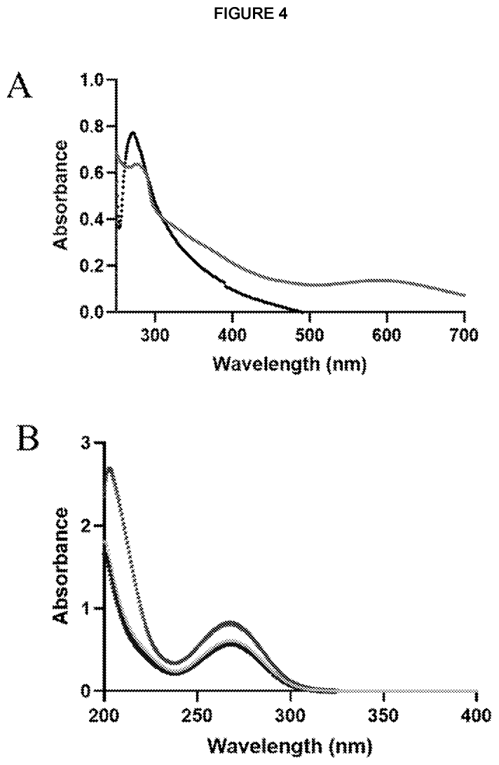

The UV spectra of the Bis-Fe₃O₄@Au-NPs (bispecific NPs conjugated to anti-EGFR Nmab and anti-CD3 scFv) and Nmab-Fe₃O₄@Au NPs (ie a nanoparticle comprising only anti-EGFR Nmab) are shown in FIG. 4. It is possible to determine the presence of conjugated antibodies by subtracting the amount of the conjugated antibodies that obtained after the centrifugation from the total amount of loaded antibodies before the centrifugation. The sharp peaks at approximately 260 nm determine the presence of conjugated antibodies.

### Cell Lines

A431 is a human epithelial carcinoma cell line with known overexpression of EGFR. Jurkat, clone E6-1 cells (TIB-125) is an acute T cell leukemia which is CD3 positive. Jurkat cells were purchased from American Type Culture collection (ATCC, MEL, VIC, AUS). MDA-MB 231 is an epithelial, human breast cancer cell line, which provides a cellular model for metastatic mammary adenocarcinoma. MDA-MB 468 is another epithelial, human breast cancer cell line, which provides a cellular model for metastatic mammary adenocarcinoma.

Cells were cultured in a flask containing Dulbecco's modified Eagle's medium (DMEM) low glucose medium, or RPMI-1640 for Jurkat, supplemented with 10% fetal bovine serum (FBS) and 1% penicillin/streptomycin, at 37° C. in a humidified incubator supplied with 5% CO₂. Cells were passaged once every 2-3 days depending on their growth rate. The cells were cultured to about 80 to 90% confluence before harvest. During the harvest, cells were washed with PBS followed by trypsinization with 2 ml trypsin and incubated for 10 minutes to detach the cells from the flask. The trypsin was neutralized by adding 8 ml of fresh supplemented medium, and then 2 ml of harvested cells was transferred to a new flask and resuspended with a fresh medium. A hemocytometer was used to determine the cell viability before each experiment.

Preparation of Anti-CD3 scFv

A construct for a specific His6-tagged anti-CD3 scFv antibody was designed from OKT3 mAb and expressed according to the method of a previous study (Kipriyanov et al. (1997) J Immunol Methods 15 (200): 67-77). The anti-CD3 scFv sequence was obtained from Protein Data Bank with code (1SY6). The construct included a linker peptide (Gly4Ser) 3 between light and heavy variable chains (SEQ ID NO: 2). The construct was cloned into the gWiz expression plasmid, which was transformed into competent Escherichia coli. Standard techniques for mammalian expression system using HEK 293 cells were used to produce the anti-CD3 scFv.

Antibodies were purified using His Trap columns equilibrated with PBS containing 10 mM imidazole. Purified antibody was dialyzed into PBS containing 15 mM sodium phosphate, 0.15 M NaCl, pH 7.4.

TABLE 2
Antibody and peptide sequences

| | SEQ | |
|---|---|---|
| Antibody | ID NO: | Sequence |
| V_{H} sequence of mouse | 1 | QVQLQQSGAELARPGASVKMSCKASGYT |
| anti-CD3 scFv | | FTRYMHWVKQRPGQGLEWIGYINPSRGYT |
| | | NYNQKFKDKATLTTDKSSSTAYMQLSSLTS |
| | | EDSAVYYCARYYDDHYSLDYWGQGTTLTV |
| | | SS |
| Linker for anti-CD3 | 2 | GGGGSGGGGSGGGGS |
| scFv | | |
| V_{L} sequence of mouse | 3 | DIVLTQSPAIMSASPGEKVTMTCSASSSVS |
| anti-CD3 scFv | | YMNWYQQKSGTSPKRWIYDTSKLASGVP |
| | | AHFRGSGSGTSYSLTISGMEAEDAATYYC |
| | | QQWSSNPFTFGSGTKLEINR |
| Anti-HER2 light chain | 4 | DIQMTQSPSSLSASVGDRVTITCRASQDVN |
| | | TAVAWYQQKPGKAPKLLIYSASFLYSGVPS |
| | | RFSGSRSGTDFTLTISSLQPEDFATYYCQQ |
| | | HYTTPPTFGQGTKVEIKRTVAAPSVFIFPP |
| | | SDEQLKSGTASVVCLLNNFYPREAKVQWK |
| | | VDNALQSGNSQESVTEQDSKDSTYSLSST |
| | | LTLSKADYEKHKVYACEVTHQGLSSPVTKS |
| | | FNRGEC |
| Anti-HER2 heavy | 5 | EVQLVESGGGLVQPGGSLRLSCAASGF |
| chain | | NIKDTYIHWVRQAPGKGLEWVARIYPTN |
| | | GYTRYADSVKGRFTISADTSKNTAYLQM |
| | | NSLRAEDTAVYYCSRWGGDGFYAMDY |
| | | WGQGTLVTVSSASTKGPSVFPLAPSSKS |
| | | TSGGTAALGCLVKDYFPEPVTVSWNSG |
| | | ALTSGVHTFPAVLQSSGLYSLSSVVTVP |
| | | SSSLGTQTYICNVNHKPSNTKVDKKVEP |
| | | KSCDKTHTCPPCPAPELLGGPSVFLFPPK |
| | | PKDTLMISRTPEVTCVVVDVSHEDPEVKFN |
| | | WYVDGVEVHNAKTKPREEQYNSTYRVVS |
| | | VLTVLHQDWLNGKEYKCKVSNKALPAPIEK |
| | | TISKAKGQPREPQVYTLPPSREEMTKNQV |
| | | SLTCLVKGFYPSDIAVEWESNGQPENNYK |
| | | TTPPVLDSDGSFFLYSKLTVDKSRWQQGN |
| | | VFSCSVMHEALHNHYTQKSLSLSPGK |
| Nimotuzumab heavy | 6 | QVQLQQSGAEVKKPGSSVKVSCKASGYTF |
| chain | | TNYYIYWVRQAPGQGLEWIGGINPTSGGS |
| | | NFNEKFKTRVTITADESSTTAYMELSSLRS |
| | | EDTAFYFCTRQGLWFDSDGRGFDFWGQG |
| | | TTVTVSSASTKGPSVFPLAPSSKSTSGGTA |
| | | ALGCLVKDYFPEPVTVSWNSGALTSGVHT |
| | | FPAVLQSSGLYSLSSVVTVPSSSLGTQTYI |
| | | CNVNHKPSNTKVDKKVP |
| Nimotuzumab light | 7 | DIQMTQSPSSLSASVGDRVTITCRSSQNIV |
| chain | | HSNGNTYLDWYQQTPGKAPKLLIYKVSNR |
| | | FSGVPSRFSGSGSGTDFTFTISSLQPEDIA |
| | | TYYCFQYSHVPWTFGQGTKLQITREVAAP |
| | | SVFIFPPSDEQLKSGTASVVCLLNNFYPRE |
| | | AKVQWKVDNALQSGNSQESVTEQDSKDS |
| | | TYSLSSTLTLSKADYEKHKVYACEVTHQGL |
| | | SSPVTKSFNRGEC |
| Panitumumab light | 8 | DIQMTQSPSSLSASVGDRVTITCQASQDIS |
| chain | | NYLNWYQQKPGKAPKLLIYDASNLETGVP |
| | | SRFSGSGSGTDFTFTISSLQPEDIATYFCQ |
| | | HFDHLPLAFGGGTKVEIKRTVAAPSVFIFPP |
| | | SDEQLKSGTASVVCLLNNFYPREAKVQWK |
| | | VDNALQSGNSQESVTEQDSKDSTYSLSST |
| | | LTLSKADYEKHKVYACEVTHQGLSSPVTKS |
| | | FNRGEC |
| Panitumumab heavy | 9 | QVQLQESGPGLVKPSETLSLTCTVSGGSV |
| chain | | SSGDYYWTWIRQSPGKGLEWIGHIYYSGN |
| | | TNYNPSLKSRLTISIDTSKTQFSLKLSSVTA |
| | | ADTAIYYCVRDRVTGAFDIWGQGTMVTVS |
| | | SASTKGPSVFPLAPCSRSTSESTAALGCLV |
| | | KDYFPEPVTVSWNSGALTSGVHTFPAVLQ |
| | | SSGLYSLSSVVTVPSSNFGTQTYTCNVDH |
| | | KPSNTKVDKTVERKC |
| V_{H} sequence of anti- | 10 | KYLLPTAAAGLLLLAAQPAMAQPAEVQLVE |
| CD16 scFv | | SGGGVVRPGGSLRLSCAASGFTFDDYGM |
| | | SWVRQAPGKGLEWVSGINWNGGSTGYAD |
| | | SVRFTISRDNAKNSLYLQMNS |
| V_{L} sequence of anti- | 11 | LRAEDTAVYYCARGRSLLFDYWGQGTLVT |
| CD16 scFv | | VSRSSELTQDPAVSVALGQTVRITCQGDSL |
| | | RSYYASWYQQKPGQAPVLVIYGKNNRPSG |
| | | IPDRFSGSSSGNTASLTITGAQAEDEADYY |
| | | CNSRDSSGNHVVFGGGTKLTVG |
| HIV-1 TAT cell | 12 | YGRKKRRQRRR |
| penetrating peptide | | |
| (resides 47-57 of HIV- | | |
| 1 TAT protein) | | |

Preparation of Fe₃O₄@Au NPs

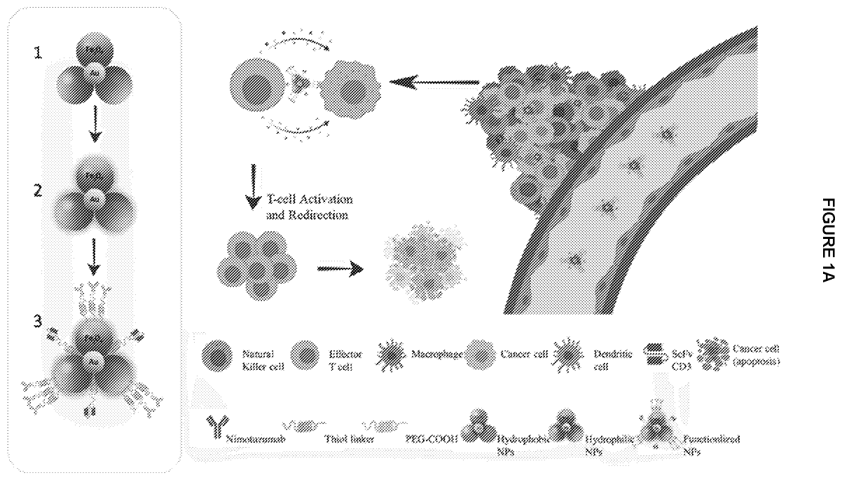

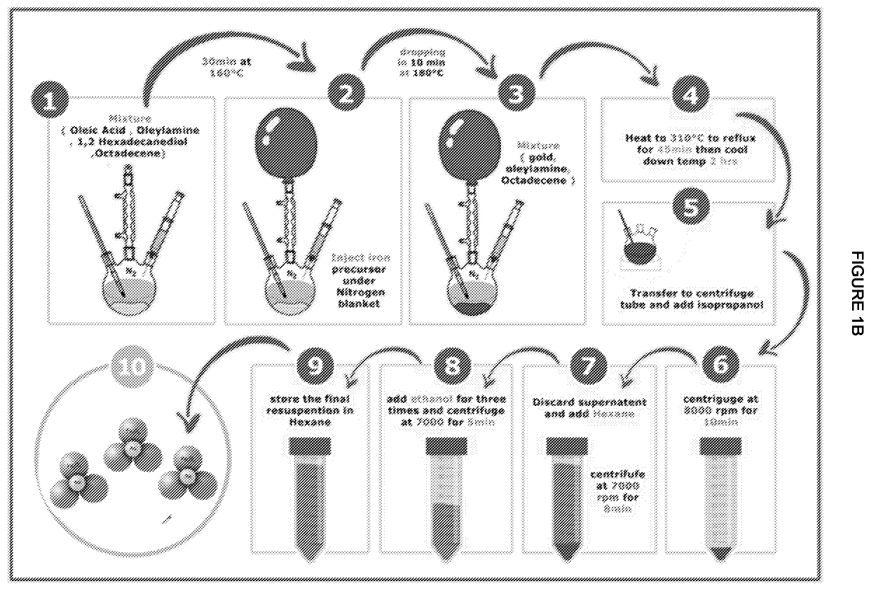

Fe₃O₄@Au NPs were synthesized according to the previously reported procedure with slight modifications (Yu, Chen et al. 2005). Fe₃O₄@Au NPs were synthesized by a thermal decomposition method under inert conditions (FIG. 1). Initially, 6 mmol oleic acid, 6 mmol oleylamine, and 10 mmol 1,2-hexadecandiol were mixed with 20 ml 1-octadecene (ODE) in a 50 ml round bottom flask equipped with thermometer, flow control adaptor, reflux condenser and rubber septum and stirred under a gentle flow of nitrogen at 160° C. for 30 min. Then under a blanket of nitrogen, 0.3 ml Fe (CO) 5 was quickly injected into the solution. After 3 min, the deaerated gold precursor solution consisting of 40 mg HAuCl₄·3H₂O, 1.5 ml oleylamine, and 5 ml ODE was dropwised into the hot solution 180° C. within 10 min to ensure fully mixed. The solution turned to dark red immediately after the injection, indicating the formation of gold nanoparticles. The solution was then slowly heated to 310° C. and refluxed for 45 min. After cooling down at room temperature, 40 ml of isopropanol was added into the solution and centrifuged at 8000 rpm for 10 min to remove large particles. The precipitate re-dispersed in hexane and centrifuged again at 7000 rpm to remove any undispersed materials. Ethanol was subsequently added into the solution and centrifuged again for three times, giving a brown dispersion. The Fe₃O₄@Au NPs were dissolved in hexane in the presence of oleylamine for further use (FIG. 1B).

Modification of Fe₃O₄@Au NPs

The transfer of hydrophobic nanoparticles into aqueous media (to improve solubility) was performed using 30 mg a, w-Bis {2-[(3-carboxy-1-oxopropyl)amino]ethyl}PEG (Mr=3,000), 2 mg of N-hydroxysuccinimide, 3 mg of dicyclohexylcarbodiimide and 1.5 mg of dopamine hydrochloride dissolved in a mixture of 1 ml dimethylformamide, 2 ml CHCl₃, and 10 mg anhydrous Na₂CO₃. The solution was stirred for 2 hours at 37° C., 5 mg Fe₃O₄@Au NPs were added, and the resulting solution was stirred overnight at 37° C. under a nitrogen blanket. The modified NPs were precipitated by adding 5 ml hexane and ethanol and collected by centrifugation at 16,500×g, surfactants and other salts were removed via dialysis (molecular mass cut off, 10 kDa) for 24 h in PBS. Finally, they were filtered through 0.22 μm sterile filter. The final iron concentration of the particles was determined by inductively coupled plasma mass spectrometry (ICP-MS).

### Statistical Analyses

Results are expressed as mean±SD. Data were analysed using GraphPad Prism (GraphPad Software, Version 9, La Jolla, CA, USA). One-way ANOVA post hoc fisher LSD was used for calculating significance were *P<0.05 was considered significant.

### Example 2: Synthesis and Characterization of Fe₃O₄@Au NPs

The 40 nm Fe₃O₄@Au NPs were synthesized via thermal decomposition at high temperature in the presence of organic solvents, which produced monodispersed highly stable NPs.

Transmission electron microscope (TEM) was used to characterize the synthesis of the flower-shaped Fe₃O₄@Au NPs. Au NPs were observed as spheres with an average core size 11 nm. The Fe₃O₄ NPs appeared as 3-4 petal shapes with a mean diameter of 14 nm.

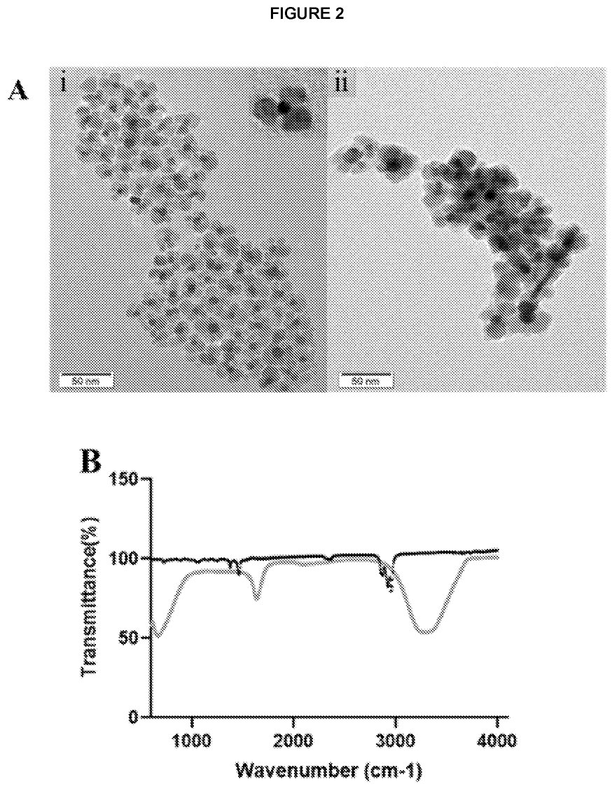

Subsequently, the organic ligands were replaced with dopamine where a 4 nm thin layer of polydopamine formed, leaving the catechol groups on the NPs surface for further conjugation. FIG. 2A shows the TEM image of 40 nm Fe₃O₄@Au NPs (due to the heavy atom effect the gold NPs appear as black). The NPs were well dispersed after ligand exchange, the hydrodynamic size of the dopamine coated NPs in solution from DLS was 136±2.49 nm which is much larger than the unmodified NPs 35.19±0.04 nm (Table 1). The size increase possibly resulted from either hydrogen bond formation or formation of a thin layer of polydopamine during the ligand exchange.

After surface modification, the zeta potential of the flower-shaped NPs after the modification was about-30 mV. FIG. 2B shows the FTIR (Fourier-transform infrared spectroscopy) spectra of Fe₃O₄@Au NPs before and after the modification. The characteristic peak at 3300 cm-1 corresponded to the catechol of dopamine. The characteristic peak C═O bands at 1653 cm-1 was an indication of successful activation of dopamine molecule on NP surfaces. NPs are commonly protected with dopamine to improve their dispersity and stability.

Medical and biological applications require the water solubility of Fe₃O₄@Au NPs. For this reason, the oleic acid coating of the NPs was modified by ligand exchange to water soluble ligands. In the present embodiment, PEG was used to align the oil phase of the NPs to the water phase, dopamine was replaced with oleic acid on the surface of iron NPs, then the amino group of dopamine was reacted with the caroboxyl group of PEG under catalysis of the EDC-Sulfo-NHS system.

To produce Fe₃O₄@Au NPs bound to nimotuzumab or to dopamine, the EDC-sulfo-NHS system was used to activate the carboxyl groups of Fe₃O₄@Au NPs modified with PEG, as aforementioned. Excess of EDC and sulfo-NHS were removed prior the Nimotuzumab addition to avoid self-crosslinking of the antibody. Following activation using the EDC-sulfo-NHS system, Nimotuzumab was conjugated to the modified Fe₃O₄@Au NPs through PEG (M_{f}=2,000) and dopamine via a condensation reaction with the formation of a peptide bond.

TABLE 1
The hydrodynamic diameter size (Z-average), PDI, Zeta potential
(ζ) and absorption maximum (λ max), and TEM size (nm) of
Fe₃O₄@Au NPs.

| | Z-ave | | ζ | λ max | TEM |
|---|---|---|---|---|---|
| NPs | (nm) | PDI | (mV) | (nm) | (nm) |
| OH—Fe₃O₄@Au | 35.19 ± 0.04 | 0.279 | — | 573 | 35 ± 2 |
| NPs | | | | | |
| Dop-PEG- | 136 ± 2.46 | 0.188 | 30.5 ± 0.7 | 390 | 39 ± 3 |
| Fe₃O₄@Au NPs | | | | | |

NP: nanoparticle format, PDI: polydispersity index, TEM: transmission electron microscope.

### Example 3: Fe₃O₄@Au NPs Conjugated to Anti-EGFR mAb (Nmab), and Anti-CD3 scFv (mAb-Fe₃O₄@Au-αCD3 NPs)

Conjugation of Fe₃O₄@Au NPs to Nimotuzumab and Anti-CD3 scFv to Generate Nmab-Fe₃O₄@Au-αCD3scFv NPs

The anti-EGFR antibody Nimotuzumab was attached to the Fe₃O₄ NPs using carbodiimide method, while anti-CD3 scFv antibody was attached to the Au NPs using a thiol linker, as briefly described herein. 1 mg of Fe₃O₄@Au NPs in methyl ester sulfonate buffer (pH 6) were mixed with 1-ethyl-3-(3-dimethylaminopropyl) carbodiimide (EDC, 1.1 mmol) and Sulfo-NHS (1 mmol) were added into the solution, to activate the carboxyl group on the PEG ligand for 30 min. After gently mixing for 30 min at room temperature, the solution was subjected to PD-10 column to remove excessive EDC and sulfo-NHS. Then Nimotuzumab 100 μg was added into the conjugate for 3 h, while gently shaking to functionalize the Fe₃O₄ side of nanoflowers. After 3 h incubation, 5 μl anti-CD3 scFv antibody was added to the mixture and the mixture is placed on a shaker for incubation overnight at room temperature to functionalize the Au side. Before immobilization of the anti-CD3 scFv, the thiol linker was added to the anti-CD3 scFv. Antibody-conjugated NPs were separated from unbound antibodies and Fe₃O₄@Au NPs using 300 K ultra-filtration.

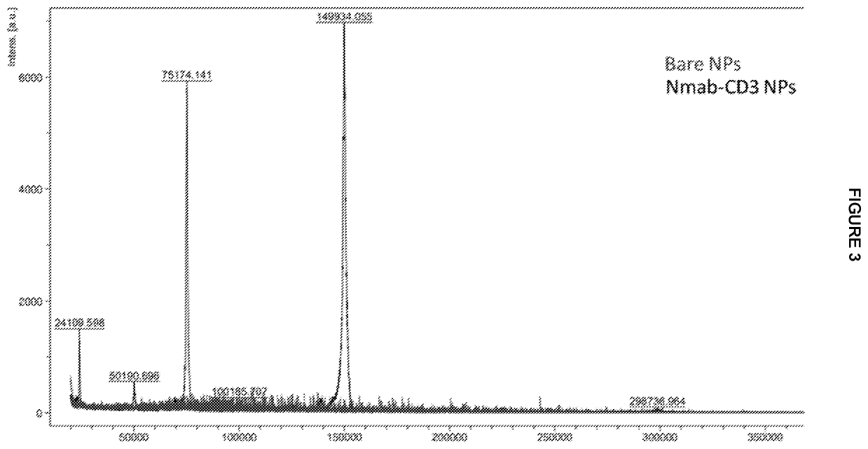

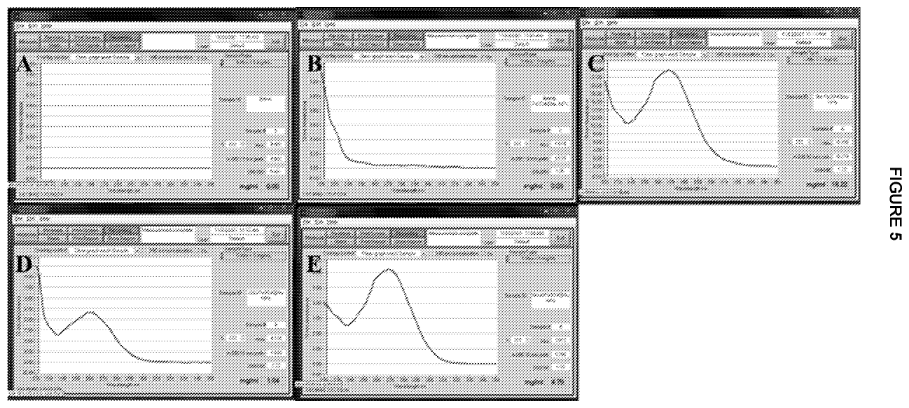

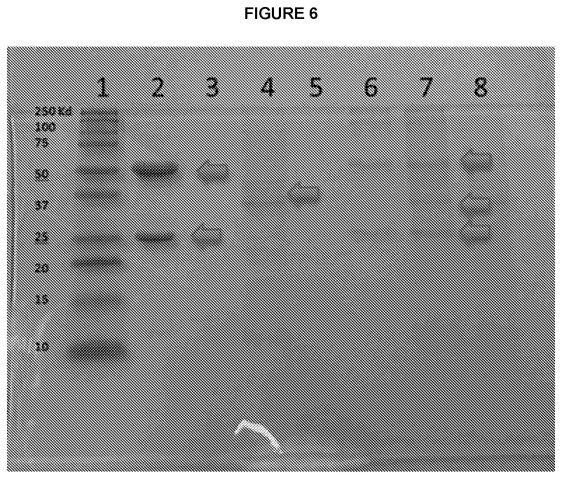

Mass spectrometry analysis of Nmab-Fe₃O₄@Au-αCD3 scFv NPs revealed a specific peak for Nimotuzumab at m/z˜148 KDa (FIG. 3), which were not detected in Au—Fe₃O₄ NPs. In addition, collected supernatants were analyzed for unbound antibody by UV-vis measurement (FIG. 4). NanoDrop 1000 spectrophotometer readings for protein confirmed the conjugation as single peak detected in the conjugated sample (FIG. 5). SDS-PAGE was also used to confirmation antibody-NP conjugation (FIG. 6).

Efficacy of Nmab-Fe₃O₄@Au-αCD3scFv NPs Assessed by Cytokine Production Using Effector: Target Cells

A431 cells were plated in triplicate in 96-well microtiter plates at 4×10³ cells/well on the day before the assay. Jurkat E6-1 cells were co-cultured with EGFR-positive A431 cells at a 5:1 effector:target (E:T) ratio in the presence of non-conjugated NPs; anti-CD3 scFv (5 μg/ml); Nmab-Fe₃O₄@Au NPs; or Bis-Fe₃O₄@Au NPs (bispecific NPs conjugated to Nmab and αCD3 scFv). After 24 hours, aliquots of the culture supernatants were harvested and the level of IL-2 were measured by using a commercially available ELISA test kit (Sydney, NSW, AUS). The samples were analyzed with a plate reader.

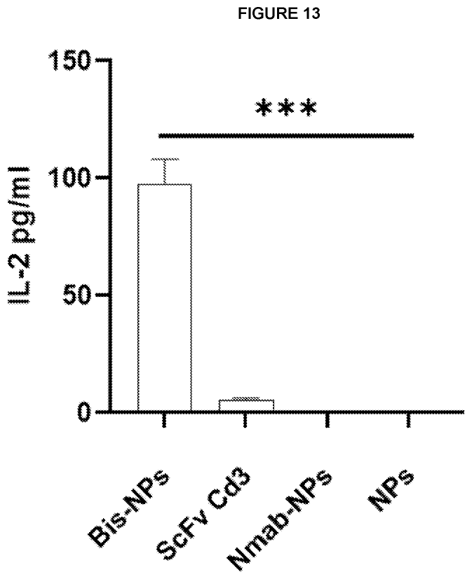

FIG. 13 shows the production of IL-2 (97.5±10 μg/ml) specifically induced after stimulation with Bis-Fe₃O₄@Au NPs (bispecific NPs conjugated to Nmab and αCD3 scFv) for 24 h. In contrast, NPs conjugated only to αCD3 scFv activated low level IL-2 production (5.5±0.7 pg/ml) from binding to T cells, and no IL-2 stimulation was observed after treatment with non-conjugated NPs or Nmab-Fe₃O₄@Au NPs. These results indicate that Bis-Fe₃O₄@Au NPs are more potent activator of T cells in vitro than NPs conjugated to anti-CD3 scFv alone.

Efficacy of Nmab-Fe₃O₄@Au-αCD3scFv NPs Assessed by Cytotoxicity Assay Using Target:Effector Cells

To assess the efficacy of bispecific nanoparticle binding, a cytotoxicity assay performed. A431 cells in the logarithmic growth phase with Fe₃O₄@Au NPs particles were plated in triplicate in 96-well microtiter plates at 4×10³ cells/well one day before the assay to grow adherent cells. MTT colorimetric assay used. This cell viability test was based on reduction of the tetrazolium salt MTT (3-(4,5-dimethylthiazol-2-yl-2,5-diphenyltetrazoliumbromide) by mitochondrial reductase in metabolically active cells. The cells were seeded onto 96-well culture plates at a density of 4000 cells per well in DMEM buffer at different concentrations were added. At 12 h, the medium was removed and replaced with 100 μl culture media containing 1% FBS.

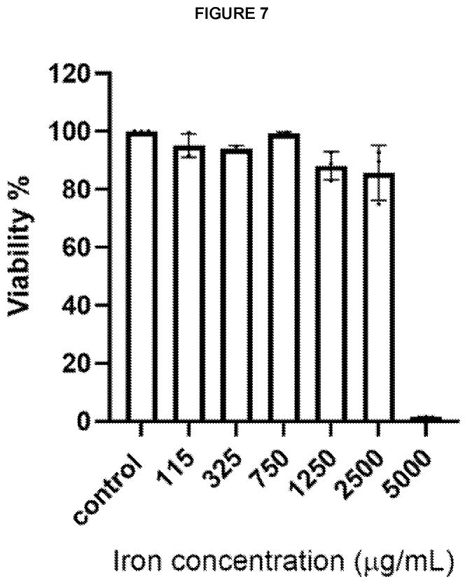

The viability of co-culture cells was not significantly affected until treated with Fe₃O₄@Au NPs with a Fe concentration >2500 μg/ml (FIG. 7).

A series of MTT assays were then performed to evaluate the cytotoxic effect of the Fe₃O₄@Au nanoparticles conjugated to Nimotuzumab and/or anti-CD3 scFv on A431 and Jurkat clone E6-1 cells, at different concentrations for five treatment groups: Group A, Nmab; Group B, Fe₃O₄@Au NPs: Group C, Nmab-Fe₃O₄@Au NPs; Group D, Nmab-Fe₃O₄@Au-αCD3 scFv NPs; Group E, cisplatin; and Group F, treatment with fresh culture medium. The treatments and effector cells were removed after 72 h incubation. Then MTT solution (5 mg/ml in PBS) was added to each well to evaluate cell viability. After 2 h at 37° C., the solution was removed. 100 μl DMSO was added to dissolve cells. After 30 min incubation at 37° C. and thorough mixing, the viability was measured by microplate reader at 570 nm. The spectrophotometer was calibrated to zero absorbance by using culture medium without cells. The cell viability was calculated and plotted against the concentration and data were evaluated using Prism 9 (GraphPad Software version 8, La Jolla, CA, USA).

The cytotoxicity of Nmab-Fe₃O₄@Au-αCD3scFv NPs (ie, nanoparticles comprising antigen binding proteins for binding EGFR and CD3), was markedly increased compared with Fe₃O₄@Au NPs.

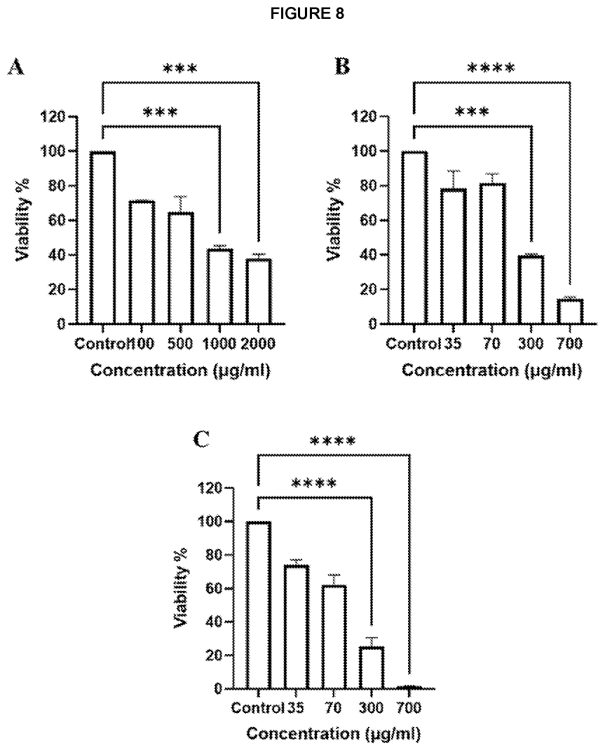

FIG. 8 shows that the viability of co-cultured cells was significantly decreased following incubation with Nmab-Fe₃O₄@Au-αCD3scFv NPs, compared with the singly-conjugated Nmab-Fe₃O₄@Au nanoparticles.

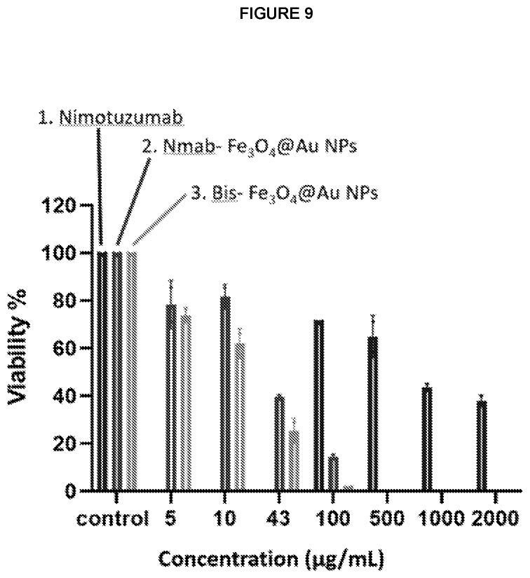

FIG. 9 similarly shows that cell viability was significantly reduced following incubation with Nmab-Fe₃O₄@Au-αCD3scFv NPs compared to Nmab (ie naked antibody at the same antibody concentration) or nanoparticles conjugated to Nimatozumab alone.

To further evaluate the cytotoxic effect of the Fe₃O₄@Au nanoparticles conjugated to Nmab and/or anti-CD3 scFv, in relation to further target cancer cells, MTT assays were performed on MDA_MB468 breast cancer cells with Jurkat effector cells, at different concentrations for three treatment groups: Group A, Free Nmab (Nimotuzumab); Group B, Nmab-Fe₃O₄@Au NPs: Group C, Nmab-Fe₃O₄@Au-αCD3scFv NPs. The Control group was treatment with fresh culture medium alone. One day before the assay, MDA-MB468 cells in the logarithmic growth phase with Fe₃O₄@Au NPs particles were plated in triplicate in 96-well microtiter plates at 4×10³ cells/well to grow adherent cells. The cells were then seeded onto 96-well culture plates at a density of 4000 cells per well in RPMI media. At 12 h, the medium was removed and replaced with 100 μl culture media containing 1% FBS. The treatments were added, then removed after 72 h incubation, and effector cells washed away with PBS. Then MTT solution (5 mg/ml in PBS) was added to each well to evaluate cell viability. After 2-4 h incubation at 37° C., the supernatant was removed. 100 μl DMSO was added to dissolve cells. After 30 min incubation at 37° C. and thorough mixing, the viability was measured by microplate reader at 570 nm. The spectrophotometer was calibrated to zero absorbance by using culture medium without cells. The cell viability was calculated and plotted against the concentration and data were evaluated using Prism 9 (GraphPad Software version 8, La Jolla, CA, USA).

The cytotoxicity of co-cultured cells treated with Nmab-Fe₃O₄@Au-αCD3scFv NPs, was markedly increased compared with Fe₃O₄@Au NPs.

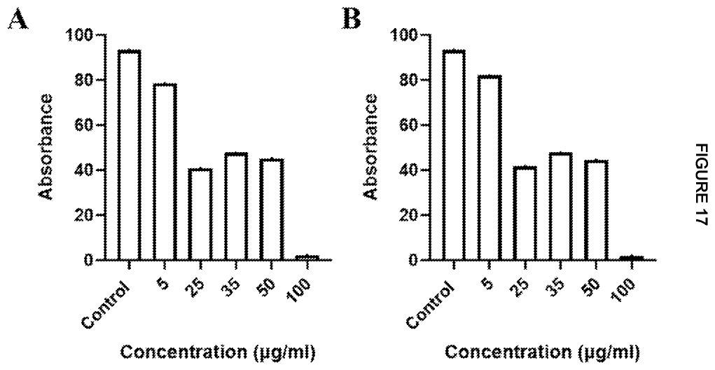

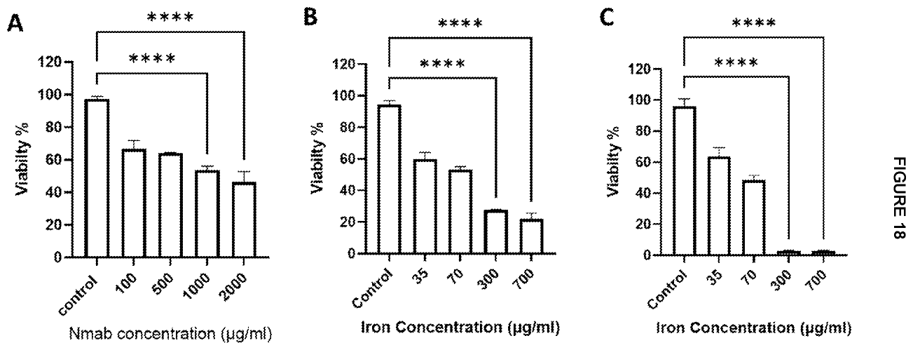

FIG. 18 shows that the viability of co-cultured cells was significantly decreased following incubation with nimotuzumab-Fe₃O₄@Au-αCD3scFv NPs, compared with the singly-conjugated Nmab-Fe₃O₄@Au nanoparticles.

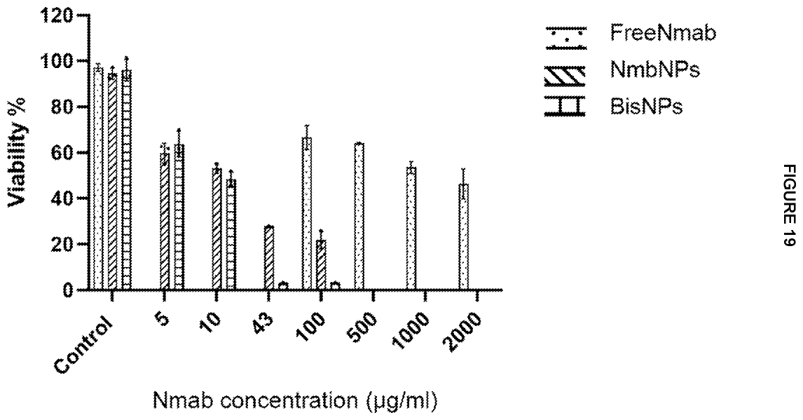

FIG. 19 similarly shows that cell viability was significantly reduced following incubation with nimotuzumab-Fe₃O₄@Au-αCD3scFv NPs compared to Nmab (ie naked antibody at the same antibody concentration) or nanoparticles conjugated to Nmab alone.

Conjugation of Fe₃O₄@Au NPs to anti-EGFR monoclonal antibody and anti-CD3 scFv to generate αCD3scFv-Fe₃O₄@Au-Nmab NPs

To evaluate bispecific nanoparticles of an alternative configuration, where the first binder was attached to the gold and the second binder was attached to the Fe₃O₄, the anti-CD3 scFv was attached to the Fe₃O₄ NPs using carbodiimide method, while anti-EGFR antibody Nimotuzumab was attached to the Au NPs using a thiol linker, as briefly described herein. 1 mg of Fe₃O₄@Au NPs in methyl ester sulfonate buffer (pH 6) were mixed with 1-ethyl-3-(3-dimethylaminopropyl) carbodiimide (EDC, 1.1 mmol) and Sulfo-NHS (1 mmol) were added into the solution, to activate the carboxyl group on the PEG ligand for 30 min. After gently mixing for 30 min at room temperature, the solution was subjected to PD-10 column to remove excessive EDC and sulfo-NHS. Then 5 μg scFv CD3 was added into the conjugate and gently shaken for 3 h to functionalise the Fe₃O₄ side of nanoflowers. After 3 h incubation, 100 μl Nmab antibody was added and the mixture and placed on a shaker for incubation overnight at room temperature to functionalize the Au side. Before immobilisation of the Nimotuzumab, the thiol linker was added to the Nmab. Antibody-conjugated NPs were separated from unbound antibodies and Fe₃O₄@Au NPs using 300 K ultra-filtration.

Efficacy of αCD3scFv-Fe₃O₄@Au-Nmab NPs Assessed by Cytotoxicity Assay Using Target:Effector Cells

To assess the efficacy of bispecific nanoparticle binding, a cytotoxicity assay performed. One day before the assay, A431 cells in the logarithmic growth phase were plated in triplicate in 96-well microtiter plates at 4×10³ cells/well to grow adherent cells. MTT colorimetric assay used as described herein. The cells were seeded onto 96-well culture plates at a density of 4000 cells per well in RPMI media. At 12 h, the medium was removed and replaced with 100 μl culture media containing 1% FBS. A series of MTT assays were then performed to evaluate the cytotoxic effect of the αCD3scFv-Fe₃O₄@Au-Nmab nanoparticles at different concentrations of Nmab or iron, compared to a Control group treated with fresh culture medium. The treatments were removed after 72 h incubation, and effector cells washed away with PBS. Then MTT solution (5 mg/ml in PBS) was added to each well to evaluate cell viability. After 2-4 h at 37° C., the MTT solution was removed. 100 μl DMSO was added to dissolve cells. After 30 min incubation at 37° C. and thorough mixing, the viability was measured by microplate reader at 570 nm. The spectrophotometer was calibrated to zero absorbance by using culture medium without cells. The cell viability was calculated and plotted against the concentration and data were evaluated using Prism 9 (GraphPad Software version 8, La Jolla, CA, USA).

The cytotoxicity of co-cultured cells treated with αCD3scFv-Fe₃O₄@Au-Nmab NPs, was markedly increased compared with no treatment (Control).

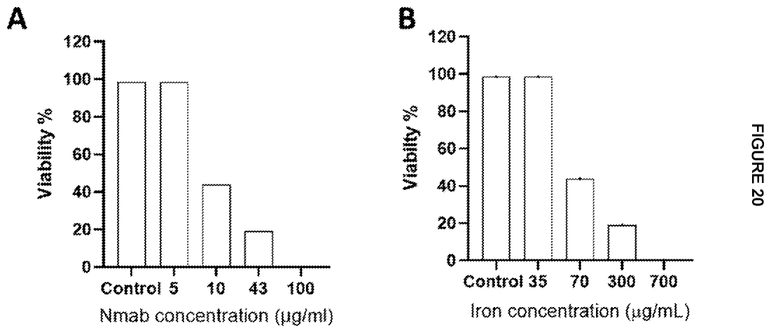

FIG. 20 shows that the viability of co-cultured cells was significantly decreased following incubation with αCD3scFv-Fe₃O₄@Au-Nmab NPs, compared with the Control.

Cellular Uptake of Fe₃O₄@Au NPs

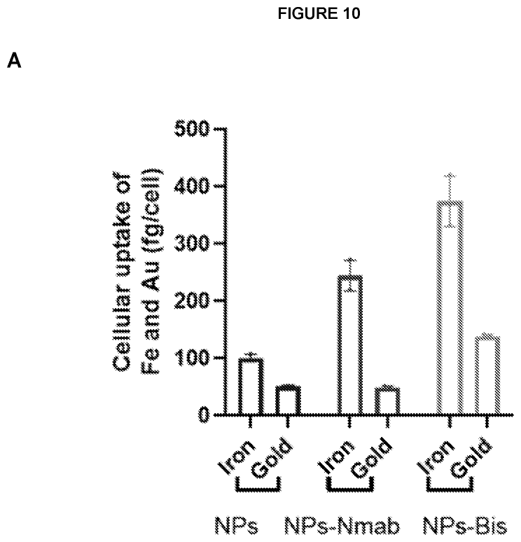

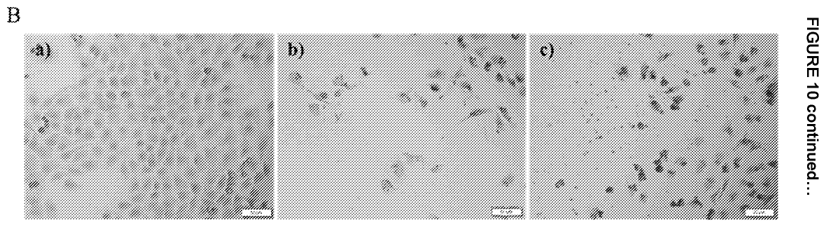

Cellular uptake of functionalized Fe₃O₄@Au NPs into cancer cell lines was demonstrated (FIG. 10). Intracellular iron and gold content was quantitatively determined by ICP-MS after incubation with 50 μg/ml iron for 12 hrs. Compared with untreated cells, the iron concentration of cell treated with Bis-Fe₃O₄@Au NPs was significantly increased to 373.92±36.1 fg/cell after incubation (FIG. 10A). Prussian blue staining (FIG. 10B) was used to qualitatively determine the iron content inside co-culture Jurkat and A431 cells treated with Bis-Fe₃O₄@Au NPs (bispecific NPs conjugated to Nmab and αCD3scFv) or Nmab-Fe₃O₄@Au NPs, or untreated.

### Example 4: Fe₃O₄@Au NPs Conjugated to Anti-HER2 mAb (Trastuzumab), and Anti-CD3 scFv (mAb-Fe₃O₄@Au-αCD3 NPs)

Preparation of Fe₃O₄@Au NPs

Fe₃O₄@Au NPs were synthesized by a thermal decomposition method according to the procedure described above with slight modifications. Briefly, 6 mmol oleic acid, 6 mmol oleylamine, and 10 mmol 1,2-hexadecandiol were mixed with 20 ml 1-octadecene (ODE) in 50 ml round bottom flask equipped with thermometer, flow control adaptor, reflux condenser, rubber septum and stirred under a gentle flow of nitrogen at 160° C. for 30 min. Then under a blanket of nitrogen, 0.3 ml Fe(CO) 5 was quickly injected into the solution. After 3 min, the deaerated gold precursor solution consisting of 40 mg HAuCl₄·3H2O, 1.5 ml oleylamine, and 5 ml ODE was added dropwise into the hot solution 180° C. within 10 min to ensure fully mixed. The solution turned to dark red immediately after the injection, indicating the formation of gold nanoparticles. The solution was then slowly heated to 310° C. and refluxed for 45 min. After cooling down at the room temperature, 40 mL of isopropanol was added into the solution and centrifuged at 8000 rpm for 10 min to remove large particles. The precipitate re-dispersed in hexane and centrifuged again at 7000 rpm to remove any undispersed materials. Ethanol was subsequently added into the solution and centrifuged again for three times, giving a brown dispersion. The Fe₃O₄@Au NPs were stored in hexane in the presence of oleylamine for further use.

Modification of Fe₃O₄@Au NPs to Improve Solubility

In order to improve the solubility of iron gold NPs, the transfer of hydrophobic nanoparticles into aqueous media was performed using a modified protocol. Briefly, 30 mg a, w-Bis {2-[(3-carboxy-1-oxopropyl)amino]ethyl}PEG (Mr=3,000), 2 mg of N-hydroxysuccinimide, 3 mg of dicyclohexylcarbodiimide and 1.5 mg of dopamine hydrochloride were dissolved in a mixture of 1 ml dimethylformamide, 2 ml CHCl₃, and 10 mg anhydrous Na₂CO₃. The solution was stirred for 2 hours at 37° C., 5 mg Fe₃O₄@Au NPs were added, and the resulting solution was stirred overnight at 37° C. under a N₂ flow. The modified NPs were precipitated by adding 5 mL hexane and ethanol and collected by centrifugation at 16,500×g, Surfactants and other salts were removed via dialysis (molecular mass cut off, 10 kDa) for 24 h in PBS. Finally, they were filtered through 0.22 μm sterile filter. The final iron concentration of the particles was determined by inductively coupled plasma mass spectrometry (ICP-MS).

Conjugation of Fe₃O₄@Au NPs to Monoclonal Antibody Trastuzumab (Tmab) and Anti-CD3 scFv Antibody

Trastuzumab (Tmab) was attached to the Fe₃O₄ NPs using carbodiimide method. While anti-CD3 scFv antibody was attached to the Au NPs using thiol linker as briefly described here. Firstly, 1 mg of Fe₃O₄@Au NPs in methyl ester sulfonate buffer (pH 6) were mixed with 1-ethyl-3-(3-dimethylaminopropyl) carbodiimide (EDC, 1.1 mmol) and Sulfo-NHS (1 mmol) were added into the solution, to activate the carboxyl group on the PEG ligand for 30 min. After gentle stirring for 30 min at room temperature, the solution was subjected to PD-10 desalting column to remove excessive EDC and sulfo-NHS. Then, Tmab (200 μg) was added into the mixture and stirred for 3 h gently to functionalize the Fe₃O₄ side of the nanoflowers. After 3 h of incubation, 5 μl of anti-CD3 scFv antibody was added, and the mixture was placed on a shaker for incubation overnight at room temperature to functionalize the Au side. Before adding the anti-CD3 scFv to the NPs mixture, the thiol linker was added to the anti-CD3 scFv. Antibody-conjugated NPs were separated from unbound antibodies and Fe₃O₄@Au NPs using 300K ultra-filtration centrifuged at 1000 rpm for 5 mins. The collected supernatants were analysed for unbound antibody by absorbance measurement using a Shimadzu 2600 UV-Vis spectrophotometer (Shimadzu, Japan). The binding efficiency was calculated as the difference between the initial addition and the amount of unbound antibody determined in the supernatants.

Efficacy of Tmab-Conjugated Fe₃O₄@Au NPs Assessed by Cytotoxicity Assay Using Target:Effector Cells

SkBr3 cells were seeded in 96-well plates (5000 cells/well). The plates were incubated with 5% CO₂ and cultivated overnight at 37° C. to promote adherence. Jurkat cells were added at a ratio of 5:1 effector (Jurkat): target (SkBr3) cells. Antibody-conjugated nanoparticles were added at various concentrations. The target SkBr3 cells and RPMI-1640 complete medium group served as controls. Following cultivation for 72 h, the effector cells were removed with washing and MTT was added for further incubation for 2 h. The supernatant was discarded and 100 μl dimethyl sulfoxide was added. After thorough mixing, the absorbance value of each well was measured using a Perkin Elmer VictorX plate reader at 570 nm. Percent viability was then calculated as [SkBr3 (treated cells)−background]/[SkBr3 (untreated cells)-background]×100. (where SkBr3 is the absorbance at 570 mm).

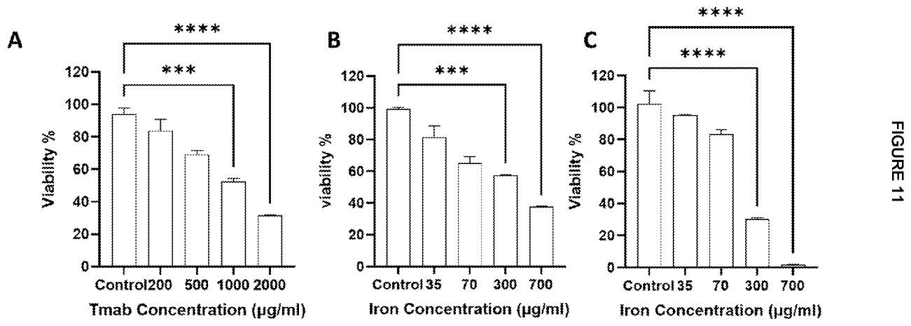

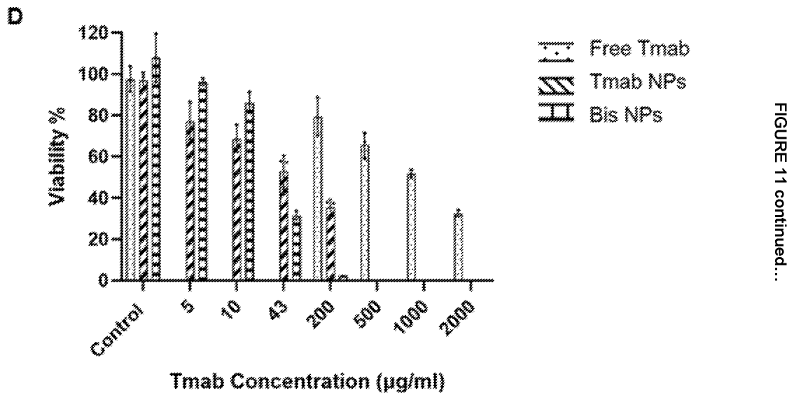

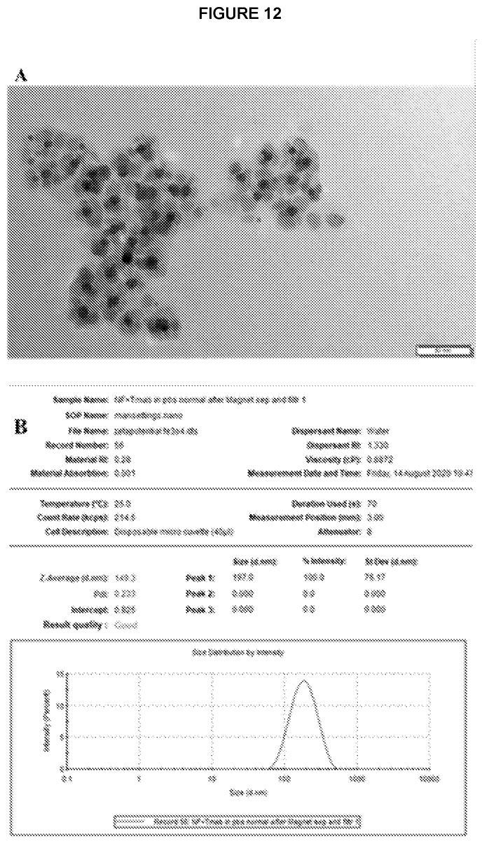

To assess the efficacy of bispecific NP binding, a cytotoxicity assay was performed in co-culture of SkBr3 and Jurkat cells at ratio 1:5 (Target:Effector) with different concentrations of Bis-Tmab-Fe₃O₄@Au-αCD3scFv (Bispecific NPs conjugated to Tmab and αCD3scFv) (FIG. 11A-C) and Tmab-Fe₃O₄@Au (FIG. 11D) NPs (NPs conjugated to Tmab alone). Both Bis-Tmab-Fe₃O₄@Au-CD3 and Tmab-Fe₃O₄@Au induced cytotoxicity of the target cells (SkBr3 cells) by effector cells (Jurkat cells). Bis-Tmab-Fe₃O₄@Au-CD3 NPs showed significantly higher cytotoxicity than Tmab-Fe₃O₄@Au NPs.

To further evaluate the cytotoxic effect of the Fe₃O₄@Au nanoparticles conjugated to Tmab and/or anti-CD3 scFv, in relation to further target cancer cells, MTT assays were performed on MDA-MB231 breast cancer cells with Jurkat effector cells, at different concentrations for three treatment groups: Group A, Free Tmab; Group B, Tmab-Fe₃O₄@Au NPs: Group C, Tmab-Fe₃O₄@Au-αCD3scFv NPs. The Control group was treatment with fresh culture medium alone. One day before the assay, MDA-MB231 cells in the logarithmic growth phase with Fe₃O₄@Au NPs particles were plated in triplicate in 96-well microtiter plates at 4×10³ cells/well to grow adherent cells. The cells were then seeded onto 96-well culture plates at a density of 4000 cells per well in RPMI media. At 12 h, the medium was removed and replaced with 100 μl culture media containing 1% FBS. The treatments were added, then removed after 72 h incubation, and effector cells washed away with PBS. Then MTT solution (5 mg/ml in PBS) was added to each well to evaluate cell viability. After 2-4 h at 37° C., the solution was removed. 100 μl DMSO was added to dissolve cells. After 30 min incubation at 37° C. and thorough mixing, the viability was measured by microplate reader at 570 nm. The spectrophotometer was calibrated to zero absorbance by using culture medium without cells. The cell viability was calculated and plotted against the concentration and data were evaluated using Prism 9 (GraphPad Software version 8, La Jolla, CA, USA).

The cytotoxicity of co-cultured cells treated with Tmab-Fe₃O₄@Au-αCD3scFv NPs, was markedly increased compared with Fe₃O₄@Au NPs.

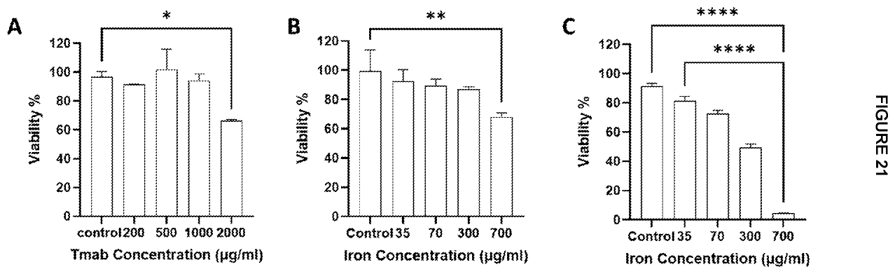

FIG. 21 shows that the viability of co-cultured cells was significantly decreased following incubation with bispecific Tmab-Fe₃O₄@Au-αCD3scFv NPs, compared with the singly-conjugated Tmab-Fe₃O₄@Au nanoparticles.

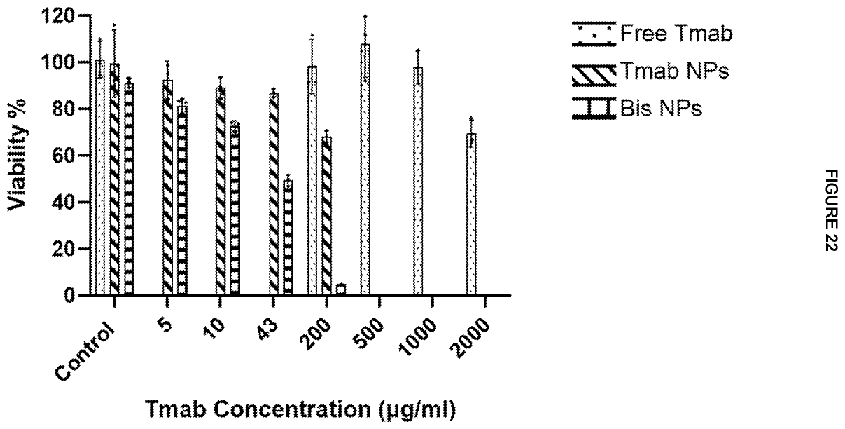

FIG. 22 similarly shows that cell viability was significantly reduced following incubation with Tmab-Fe₃O₄@Au-αCD3scFv NPs compared to free Tmab (ie naked antibody at the same antibody concentration) or nanoparticles conjugated to Tmab alone.

### Example 5: Fe₃O₄@Au NPs Conjugated to Anti-EGFR mAb (Panitumumab, Pmab), and Anti-CD3 scFv (mAb-Fe₃O₄@Au-αCD3 scFv NPs)

Conjugation of Fe₃O₄@Au NPs to Monoclonal Antibody Pmab and Anti-CD3 scFv Antibody

To assess the efficacy of bispecific nanoparticles using an alternative anti-EGFR binder, anti-EGFR monoclonal antibody Panitumumab was attached to the Fe₃O₄ NPs using carbodiimide method, while anti-CD3 scFv antibody was attached to the Au NPs using a thiol linker, as briefly described herein. 1 mg of Fe₃O₄@Au NPs in methyl ester sulfonate buffer (pH 6) were mixed with 1-ethyl-3-(3-dimethylaminopropyl) carbodiimide (EDC, 1.1 mmol) and Sulfo-NHS (1 mmol) were added into the solution, to activate the carboxyl group on the PEG ligand for 30 min. After gently mixing for 30 min at room temperature, the solution was subjected to PD-10 column to remove excessive EDC and sulfo-NHS. Then, 100 μg Panitumumab was added into the conjugate and shaken for 3 h, while gently shaking to functionalize the Fe₃O₄ side of nanoflowers. After 3 h incubation, 5 μl anti-CD3 scFv antibody was added and the mixture and placed on a shaker for incubation overnight at room temperature to functionalize the Au side. Before immobilization of the anti-CD3 scFv, the thiol linker was added to the anti-CD3 scFv. Antibody-conjugated NPs were separated from unbound antibodies and Fe₃O₄@Au NPs using 300 K ultra-filtration.

Efficacy of Pmab-Conjugated Fe₃O₄@Au NPs Assessed by Cytotoxicity Assay Using Target:Effector Cells

To assess the efficacy of bispecific nanoparticle binding, a cytotoxicity assay performed. One day before the assay, A431 cells in the logarithmic growth phase with Fe₃O₄@Au NPs particles were plated in triplicate in 96-well microtiter plates at 4×10³ cells/well to grow adherent cells. MTT colorimetric assay used. This cell viability test was based on reduction of the tetrazolium salt MTT (3-(4,5-dimethylthiazol-2-yl-2,5-diphenyltetrazoliumbromide) by mitochondrial reductase in metabolically active cells. The cells were seeded onto 96-well culture plates at a density of 4000 cells per well in DMEM media. At 12 h, the medium was removed and replaced with 100 μl culture media containing 1% FBS.

MTT assays were then performed to evaluate the cytotoxic effect of the Pmab-Fe₃O₄@Au-αCD3scFv nanoparticles on A 431 and Jurkat cells at different concentrations of Pmab or iron. The Control group was treatment with cell medium alone. The treatments were removed after 72 h incubation, and effector cells washed away with PBS. Then MTT solution (5 mg/ml in PBS) was added to each well to evaluate cell viability. After 2-4 h at 37° C., the solution was removed. 100 μl DMSO was added to dissolve cells. After 30 min incubation at 37° C. and thorough mixing, the viability was measured by microplate reader at 570 nm. The spectrophotometer was calibrated to zero absorbance by using culture medium without cells. The cell viability was calculated and plotted against the concentration and data were evaluated using Prism 9 (GraphPad Software version 8, La Jolla, CA, USA).

The cytotoxicity of co-cultured cells treated with Pmab-Fe₃O₄@Au-αCD3scFv NPs, was markedly increased compared with Fe₃O₄@Au NPs.

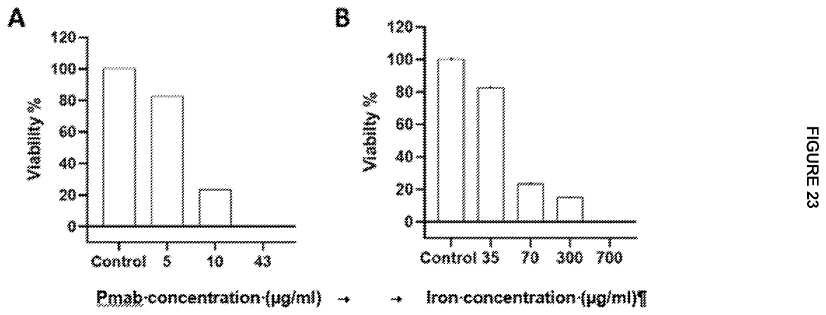

FIG. 23 shows that the viability of co-cultured cells was significantly decreased following incubation with bispecific Pmab-Fe₃O₄@Au-αCD3scFv NPs, compared with the singly-conjugated Pmab-Fe₃O₄@Au nanoparticles.

### Example 6: Bispecific Fe₃O₄@Au NPs Conjugated to Cell-Penetrating Peptide (CPP)

Bispecific Fe₃O₄@Au NPs conjugated to a cell-penetrating peptide (mAb-Fe₃O₄@Au NPs-αCD3/CPP NPs) were synthesised by conjugating Fe₃O₄@Au NPs to an anti-EGFR monoclonal antibody (Nmab), anti-CD3 scFv, and CPP.

### HIV-TAT Cell Penetrating Peptide PEGlyation (CPP-PEG-SH)

The HIV-TAT (residues 47-57) cell penetrating peptide was dissolved in NaHCO₃ 0.1 M pH 8.0 to a concentration of 1 mg/ml (641 M). At a 4:1 NHS-linker/CPP molar ratio, a 10 mg/mL (2 mM) NHS-PEG-SH (5 kDa) solution in NaHCO₃ 0.1 M pH 8.0 was added to the HIV-TAT peptide and incubated overnight at 4° C. Unreacted CPP was removed by centrifugation through 3 kDa cut off filters and the PEGylated CPP was buffer exchanged to phosphate buffered saline 0.01 M pH 7.4 with 1 mM EDTA.

### Nanoparticle Surface Functionalization

The anti-EGFR antibody Nimotuzumab was attached to the Fe₃O₄ NPs using carbodiimide method, while anti-CD3 scFv antibody and the HIV-TAT CPP were attached to the Au NPs using a thiol linker, as briefly described herein. 1 mg of Fe₃O₄@Au NPs in methyl ester sulfonate buffer (pH 6) were mixed with 1-ethyl-3-(3-dimethylaminopropyl) carbodiimide (EDC, 1.1 mmol) and Sulfo-NHS (1 mmol) were added into the solution, to activate the carboxyl group on the PEG ligand for 30 min. After gently mixing for 30 min at room temperature, the solution was subjected to PD-10 column to remove excessive EDC and sulfo-NHS. Then 100 μg Nimotuzumab was added into the conjugate and incubated for 3 h, while gently shaking to functionalize the Fe₃O₄ side of nanoflowers. After 3 h incubation, 5 μl anti-CD3 scFv antibody was added and the mixture and placed on a shaker for incubation overnight at room temperature to functionalize the Au side. Before immobilization of the anti-CD3 scFv, the thiol linker was added to the anti-CD3 scFv. The PEGylated CPP was added after buffer exchanged to PBS 0.01 M pH 7.4 with 1 mM EDTA. Antibody-conjugated NPs were separated from unbound antibodies and non-conjugated Fe₃O₄@Au NPs using 300 K ultra-filtration.

Efficacy of Nmab-Fe₃O₄@Au-αCD3 scFv/CPP NPs Assessed by Cytotoxicity Assay Using Target:Effector Cells

To assess the efficacy of CPP bispecific nanoparticle binding, a cytotoxicity assay performed. A431 cells in the logarithmic growth phase with Fe₃O₄@Au NPs particles were plated in triplicate in 96-well microtiter plates at 4×10³ cells/well one day before the assay, to grow adherent cells. MTT colorimetric assay used. This cell viability test was based on reduction of the tetrazolium salt MTT (3-(4,5-dimethylthiazol-2-yl-2,5-diphenyltetrazoliumbromide) by mitochondrial reductase in metabolically active cells. The cells were seeded onto 96-well culture plates at a density of 4000 cells per well in DMEM buffer at different concentrations were added. At 12 h, the medium was removed and replaced with 100 μl culture media containing 1% FBS. The A431 cells were designated as the target cells and Jurkat E6-1 were considered as effector cells. Jurkat E6-1 cells were added in 5:1 effector:target (E:T) ratio on A431 cells in the presence or absence of Nmab-Fe₃O₄@Au-αCD3 scFv/CPP NPs. Untreated cells were chosen as controls. Following cultivation for 72 h, the effector cells were washed away with PBS and MTT was added for further incubation for 2-4 h. The supernatant was discarded and 100 μl dimethyl sulfoxide was added. After thorough mixing, the absorbance value of each well was measured using a Perkin Elmer VictorX plate reader (Melbourne, VIC, Australia) at 570 nm. Percent viability was then calculated as [A431 (treated cells)−background]/[A431 (untreated cells)−background]×100. (where A431 is the absorbance at 570 mm).

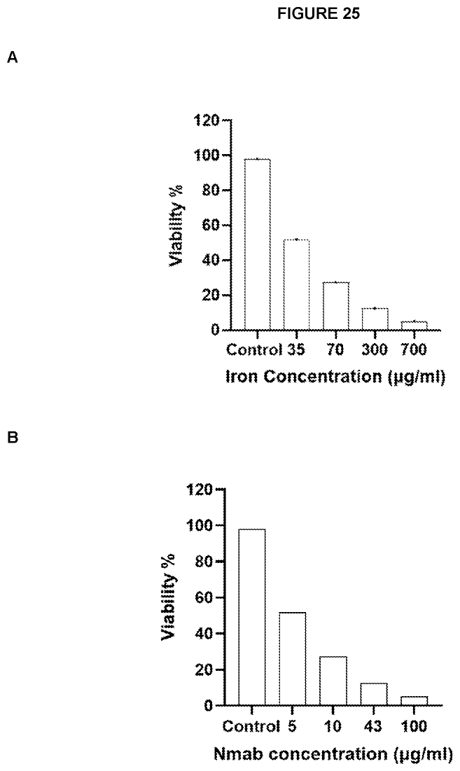

FIG. 25 shows that the viability of co-cultured A431 and Jurkat cells was significantly decreased following incubation with ≥70 μg/ml iron concentration of Nmab-Fe₃O₄@Au-αCD3scFv/CPP NPs (FIG. 25A); and with ≥10 μg/ml Nmab concentration of Nmab-Fe₃O₄@Au-αCD3scFv/CPP NPs (FIG. 25B).

The cytotoxicity of co-cultured cells treated with Nmab-Fe₃O₄@Au-αCD3 scFv/CPP NPs, was markedly increased compared with Nmab-Fe₃O₄@Au-αCD3 scFv NPs without CPP.

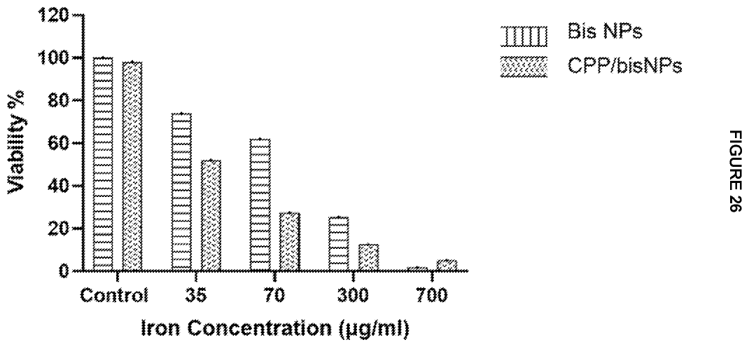

FIG. 26 shows that the viability of co-cultured cells was significantly decreased following incubation with bispecific Nmab-Fe₃O₄@Au-αCD3 scFv/CPP NPs, compared with Nmab-Fe₃O₄@Au-αCD3 scFv NPs without CPP.

### Example 7: Preparation of Bi-Specific Silver-Iron Nanoparticles (Fe₃O₄@Ag NPs)

Fe₃O₄@Ag NPs were synthesized by a thermal decomposition method according to the previously reported procedures with slight modifications. Briefly, 6 mmol oleic acid, 6 mmol oleylamine, and 10 mmol 1,2-hexadecandiol were mixed with 20 ml 1-octadecene (ODE) in 50 ml round bottom flask equipped with thermometer, flow control adaptor, reflux condenser, rubber septum and stirred under a gentle flow of nitrogen at 120° C. for 30 min. Then under a blanket of nitrogen, 0.3 ml Fe(CO)₅ solution was quickly injected into the solution. After 5 min, the deaerated silver precursor solution consisting of 35 mg AgNO₃, 1 ml oleylamine, and 5 ml toluene solution was added dropwise into the hot solution 180° C. within 10 min to ensure fully mixed. The solution turned to dark brown immediately after the injection, indicating the formation of silver nanoparticles. The solution was then slowly heated to 205° C. and refluxed for 90 min. After cooling down at the room temperature, 40 mL of isopropanol was added into the solution and centrifuged at 8000 rpm for 10 min to remove large particles. The precipitate re-dispersed in hexane and centrifuged again at 7000 rpm to remove any undispersed materials. Ethanol was subsequently added into the solution and centrifuged again for three times, giving a brown dispersion. The Fe₃O₄@Ag NPs were stored in hexane in the presence of 0.5 μl oleylamine for further use, including for conjugation to antigen binding proteins, as described elsewhere herein.

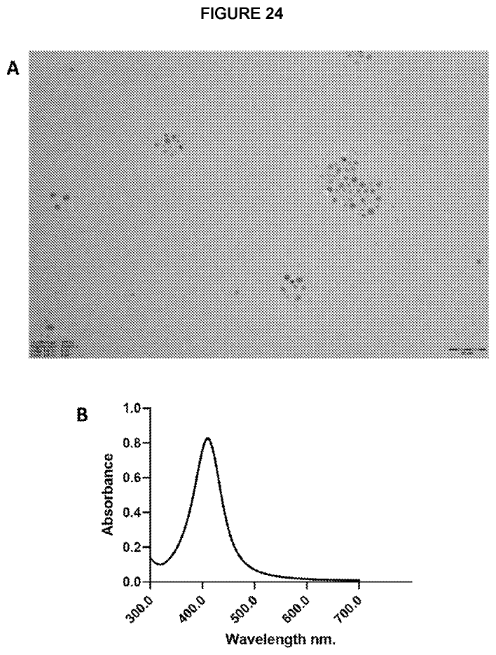

Transmission electron microscope (TEM) was used to characterize the synthesis of the Fe₃O₄@Ag NPs (FIG. 24A). Ag NPs were observed with an average core size 9 nm. The Fe₃O₄ NPs appeared as dumbbell-shaped. FIG. 24B shows the UV-Vis spectra of Fe₃O₄@Ag NPs, with a peak at 409 nm. The hydrodynamic size of NPs was 8.23±0.03 nm (Table 3).

TABLE 3
The hydrodynamic diameter size (Z-average), PDI, Zeta potential
(ζ) and absorption maximum (λ max), and TEM size (nm) of
Fe₃O₄@Ag NPs.

| | Z-ave | | λ max | TEM |
|---|---|---|---|---|
| NPs | (nm) | PDI | (nm) | (nm) |
| Fe₃O₄@Ag NPs | 8.2999 ± 0.03 | 0.058 | 409 | 9 |

NP: nanoparticle format, PDI: polydispersity index, TEM: transmission electron microscope.

### Example 8: Dumbbell-Like Fe₃O₄@Au NPs Conjugated to Anti-EGFR mAb (Nmab) and Anti-CD3 scFv (mAb-Fe₃O₄@Au-αCD3 NPs)

TEM was used to confirm the morphology as well as the synthesis of the dumbbell-like Fe₃O₄@Au NPs. Methods for making dumbbell-shaped nanoparticles are known to the skilled person and include minor modification to the methods used for generating flower-shaped nanoparticles. In brief, reducing the molar ration of iron pentacarbonyl in the preparation of nanoparticles facilitates generation of a dumbbell-shape compared to a flower-shaped nanoparticle.

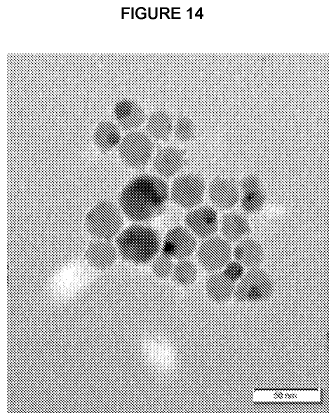

As shown in FIG. 14, Au NPs were observed as spheres with an average core size of ˜5 nm (shown as black in colour due to heavy atom effect), whereas the Fe₃O₄ NPs show a diameter of ˜10 nm. Cytotoxicity assays confirmed the viability of co-culture cells was significantly decreased following incubation with dumbbell-like Bis-Nmab-Fe₃O₄@Au-αCD3scFv NPs (bispecific NPs conjugated with Nmab and anti-CD3 scFv, when compared with spherical Nmab-Fe₃O₄@Au NPs or bare non-conjugated NPs with a ≥300 μg/ml iron (FIG. 15).

### Example 9: Spherical Au NPs Conjugated to Anti-EGFR mAb (Nmab), and Anti-CD3 scFv (Nmab-Fe₃O₄@Au-CD3 NPs)

### Preparation of Spherical Au NPs

Briefly, 150 μL of 22 mM trisodium citrate, 50 μL of 2.5 mM tannic acid, and 50 μL of 150 mM potassium carbonate were added to the conical flask containing 30 mL of Milli-Q water and stirred vigorously at 60 0C for 5 min. Then, 1.25 mL of 12.5 mM HAuCL4 solution was added slowly and stirred for 2 min at 60° C. The temperature was reduced to 40° C. as soon as the color changed from light yellow to blackish color and was left to stir gently for 15 min. The Au NPs cooled at room temperature and were then stored at 40° C.

Conjugation of @Au NPs to Monoclonal Antibody Anti-EGFR mAb (Nmab) and Anti-CD3 scFv Antibody

For PEGylation of Au NPs, 1 ml (100 μg) of Au NPs and 20 UL of Thiol-PEG-NHS linker stock solutions (10 mg/ml) were mixed in a small tube and incubated overnight in the cold room under gentle rotation. The anti-CD3 scFv (5 μL) and Nmab (200 μg) antibodies where then to the NPs mixture, 10 μL and 20 μL respectively. Antibody-conjugated NPs were separated from unbound antibodies and Au NPs using ultra filtration tube.

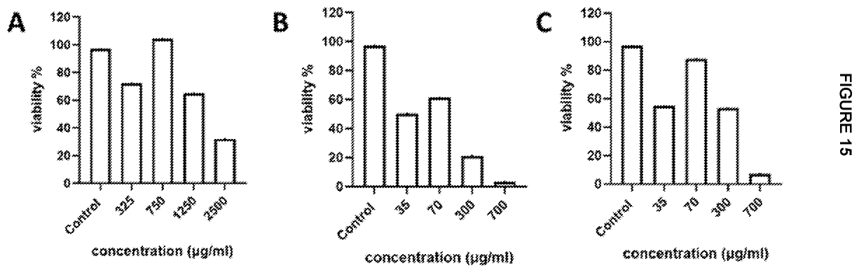

TEM was used to confirm the morphology as well as the synthesis of the spherical Au NPs. As shown in FIG. 16A, Au NPs were observed as spheres with an average size of ˜ 17 nm, black in color due to heavy atom effect.

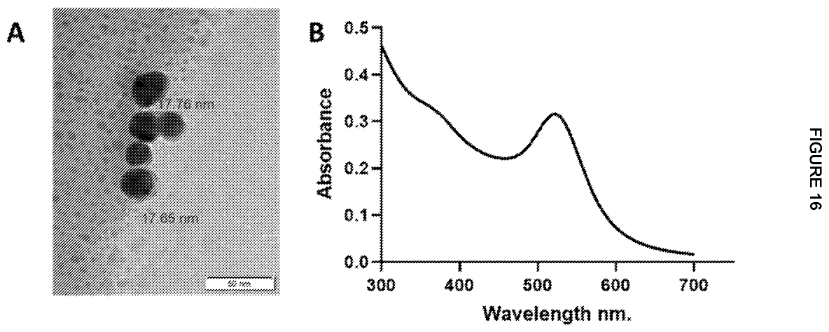

A cytotoxicity assay was performed by co-culture of A431 and Jurkat cells at ratio 1:5 (Target:Effector) treated with (A) spherical Nmab-Fe₃O₄@Au NPs, or (B) spherical Bis-Fe₃O₄@Au NPs (bispecific NPs conjugated with Nmab and anti-CD3 scFv) at different concentrations. Cell viability (measured as absorbance) was not significantly affected after incubation with Bis-Fe₃O₄@AuNPs (FIG. 17B), compared with Nmab@Au NPs (FIG. 17A). These results show that antigen binding protein-conjugated spherical gold nanoparticles did not significantly induce cell killing when compared to conjugated flower-shaped nanoparticles. The results demonstrate that flower shaped iron gold NPs are likely to provide a superior therapeutic outcome compared to nanoparticles of alternative configurations.

### Example 10: Fe₃O₄@Au NPs Conjugated to Anti-EGFR mAb (Nmab), and Anti-CD16 scFv (mAb-Fe₃O₄@Au-αCD16 scFv NPs)

Conjugation of Fe₃O₄@Au NPs to Nimotuzumab and anti-CD16 scFv is to be performed to generate Nmab-Fe₃O₄@Au-αCD16 scFv NPs

The anti-EGFR antibody Nimotuzumab is attached to the Fe₃O₄ NPs using the aforementioned carbodiimide method, while anti-CD16 scFv antibody is attached to the Au NPs using a thiol linker, as briefly described herein. 1 mg of Fe₃O₄@Au NPs in methyl ester sulfonate buffer (pH 6) is mixed with 1-ethyl-3-(3-dimethylaminopropyl) carbodiimide (EDC, 1.1 mmol) and Sulfo-NHS (1 mmol) is added into the solution, to activate the carboxyl group on the PEG ligand for 30 min. After gently mixing for 30 min at room temperature, the solution is subjected to PD-10 column to remove excessive EDC and sulfo-NHS. Then 100 μg Nimotuzumab is added into the conjugate for 3 h, while gently shaking to functionalize the Fe₃O₄ side of nanoflowers. After 3 h incubation, 5 μl anti-CD16 scFv antibody is added to the mixture and the mixture is placed on a shaker for incubation overnight at room temperature to functionalize the Au side. Before immobilization of the anti-CD16 scFv, the thiol linker is added to the anti-CD16 scFv. Antibody-conjugated NPs are separated from unbound antibodies and non-conjugated Fe₃O₄@Au NPs using 300 K ultra-filtration.

Mass spectrometry analysis of Nmab-Fe₃O₄ @Au-αC16scFv NPs is used to reveal a specific peak for Nimotuzumab at m/z ˜148 KDa, which is not detectable in Au—Fe₃O₄ NPs. In addition, collected supernatants are analysed for unbound antibody by UV-vis measurement as described herein. NanoDrop 1000 spectrophotometer readings for protein will confirm the conjugation as single peak detected in the conjugated sample. SDS-PAGE can also be used to confirm antibody-NP conjugation.

Efficacy of Nmab-Fe₃O₄@Au-αCD3scFv NPs is to be Assessed by Cytokine Production Using Effector:Target Cells

A431 cells in the logarithmic growth phase are plated in triplicate in 96-well microtiter plates at 4×10³ cells/well on the day before the assay to grow adherent cells. Jurkat E6-1 cells are co-cultured with EGFR-positive A431 cells at a 5:1 effector:target (E:T) ratio in the presence of non-conjugated NPs; anti-CD16 scFv (5 μg/ml); Nmab-Fe₃O₄@Au NPs; or Bis-Fe₃O₄@Au NPs (bispecific NPs conjugated to Nmab and αCD16 scFv). After 24 hours, aliquots of the culture supernatants are harvested and the level of IL-2 is measured by using a commercially available ELISA test kit (Sydney, NSW, AUS). The samples are analyzed with a plate reader.

Results are expected to show the production of IL-2 specifically induced after stimulation with Bis-Fe₃O₄@Au NPs (bispecific NPs conjugated to Nmab and αCD16 scFv) for 24 h. In contrast, NPs conjugated only to αCD16 scFv is expected to activate no to low level IL-2 production from binding to T cells, and no IL-2 stimulation is expected to be observed after treatment with non-conjugated NPs or Nmab-Fe₃O₄@Au NPs. The results will indicate that Bis-Fe₃O₄@Au NPs are more potent activator of T cells in vitro than NPs conjugated to anti-CD16 scFv alone.

Efficacy of Nmab-Fe₃O₄@Au-αCD16 scFv NPs is to be Assessed by Cytotoxicity Assay Using Target:Effector Cells

To assess the efficacy of bispecific nanoparticle binding, a cytotoxicity assay is performed. A431 cells in the logarithmic growth phase with Fe₃O₄@Au NPs particles are plated in triplicate in 96-well microtiter plates at 4×10³ cells/well one day before the assay, to grow adherent cells. A MTT colorimetric assay is used. This cell viability test is based on reduction of the tetrazolium salt MTT (3-(4,5-dimethylthiazol-2-yl-2,5-diphenyltetrazoliumbromide) by mitochondrial reductase in metabolically active cells. The cells are seeded onto 96-well culture plates at a density of 4000 cells per well in DMEM buffer at different concentrations are added. At 12 h, the medium is removed and replaced with 100 μl culture media containing 1% FBS.

The viability of co-culture cells will not be significantly affected until treated with Fe₃O₄@Au NPs with a Fe concentration >2500 μg/ml.

A series of MTT assays are then performed to evaluate the cytotoxic effect of the Fe₃O₄@Au nanoparticles conjugated to Nimotuzumab and/or anti-C16 scFv on A431 and Jurkat cells, at different concentrations for five treatment groups: Group A, Nmab; Group B, Fe₃O₄@Au NPs: Group C, Nmab-Fe₃O₄@Au NPs; Group D, Nmab-Fe₃O₄@Au-αCD16 scFv NPs; Group E, cisplatin; and Group F, treatment with fresh culture medium. The treatments are removed after 72 h incubation, and effector cells washed away with PBS. Then MTT solution (5 mg/ml in PBS) is added to each well to evaluate cell viability. After 2-4 h at 37° C., the solution is removed. 100 μl DMSO is added to dissolve cells. After 30 min incubation at 37° C. and thorough mixing, the viability is measured by microplate reader at 570 nm. The spectrophotometer is calibrated to zero absorbance by using culture medium without cells. The cell viability is calculated and plotted against the concentration and data is evaluated using Prism 9 (GraphPad Software version 8, La Jolla, CA, USA).

The cytotoxicity of Nmab-Fe₃O₄@Au-αCD16scFv NPs (ie, nanoparticles comprising antigen binding proteins for binding EGFR and CD16), will be markedly increased compared with Fe₃O₄@Au NPs.

The results will show that viability of co-cultured cells is significantly decreased following incubation with Nmab-Fe₃O₄@Au-αCD16scFv NPs, compared with the singly-conjugated Nmab-Fe₃O₄@Au nanoparticles.

The results are expected to similarly show that cell viability is significantly reduced following incubation with Nmab-Fe₃O₄@Au-αCD16scFv NPs compared to Nmab (ie naked antibody at the same antibody concentration) or nanoparticles conjugated to Nimatozumab alone.

To further evaluate the cytotoxic effect of the Fe₃O₄@Au nanoparticles conjugated to Nmab and/or anti-CD16 scFv, in relation to further target cancer cells, MTT assays are performed on MDA_MB468 breast cancer cells with Jurkat effector cells, at different concentrations for three treatment groups: Group A, Free Nmab (Nimotuzumab); Group B, Nmab-Fe₃O₄@Au NPs: Group C, Nmab-Fe₃O₄@Au-αCD16scFv NPs. The Control group is treatment with fresh culture medium alone. One day before the assay, MDA-MB468 cells in the logarithmic growth phase with Fe₃O₄@Au NPs particles are plated in triplicate in 96-well microtiter plates at 4×10³ cells/well, to grow adherent cells. The cells are seeded onto 96-well culture plates at a density of 4000 cells per well in RPMI media. At 12 h, the medium is removed and replaced with 100 μl culture media containing 1% FBS. The treatments are added, then removed after 72 h incubation, and effector cells are washed away with PBS. Then MTT solution (5 mg/ml in PBS) is added to each well to evaluate cell viability. After 2-4 h at 37° C., the solution is removed. 100 μl DMSO is added to dissolve cells. After 30 min incubation at 37° C. and thorough mixing, the viability is measured by microplate reader at 570 nm. The spectrophotometer is calibrated to zero absorbance by using culture medium without cells. The cell viability is calculated and plotted against the concentration and data is evaluated using Prism 9 (GraphPad Software version 8, La Jolla, CA, USA).

The cytotoxicity of co-cultured cells treated with Nmab-Fe₃O₄@Au-αCD16 scFv NPs, will be markedly increased compared with Fe₃O₄@Au NPs.

Results will show that the viability of co-cultured cells is significantly decreased following incubation with nimotuzumab-Fe₃O₄@Au-αCD16scFv NPs, compared with the singly-conjugated Nmab-Fe₃O₄@Au nanoparticles.

Results will similarly show that cell viability is significantly reduced following incubation with nimotuzumab-Fe₃O₄@Au-αCD16scFv NPs compared to Nmab (ie naked antibody at the same antibody concentration) or nanoparticles conjugated to Nmab alone.

1. A nanoparticle bioconjugate comprising:
a 1^{st} nanoparticle to which is conjugated a 1^{st} binding protein for specifically binding a target moiety on a cancer cell, and
a 2^{nd} nanoparticle, to which is conjugated a 2^{nd} binding protein for specifically binding a target moiety on an immune cell;
wherein the nanoparticles are arranged in a dumbbell or flower-like configuration, and
wherein the bioconjugate is capable of binding a cancer cell via the 1^{st} binding protein and an immune cell via the 2^{nd} binding protein.

2. The nanoparticle bioconjugate of claim 1, wherein the 1^{st} nanoparticle is a metallic nanoparticle and the 2^{nd} nanoparticle is a magnetic nanoparticle.

3. The nanoparticle bioconjugate of claim 1, wherein the 1^{st} nanoparticle is a magnetic nanoparticle and the 2^{nd} nanoparticle is a metallic nanoparticle.

4. The nanoparticle bioconjugate of claim 1 or 2, wherein the metallic nanoparticle is magnetic or non-magnetic.

5. The nanoparticle bioconjugate of any one of claims 1 to 4, wherein the architecture of the nanoparticle bioconjugate is in the form of a central metallic nanoparticle, surrounded by 3-4 or more magnetic nanoparticles, in a flower-like configuration.

6. The nanoparticle bioconjugate of any one of claims 1 to 4, wherein the nanoparticle bioconjugate is in the form of a central magnetic nanoparticle, surrounded by 3-4 or more metallic nanoparticles, in a flower-like configuration.

7. The nanoparticle bioconjugate of any one of claims 1 to 4, wherein the nanoparticle bioconjugate comprises the 1^{st} and 2^{nd} nanoparticles in a “dumbbell-like” architecture, whereby preferably the nanoparticle conjugate comprises a metallic nanoparticle adjacent to, or in intimate contact, with a magnetic nanoparticle.

8. The nanoparticle bioconjugate of any one of claims 1 to 7, wherein the nanoparticle bioconjugate is not in the form of a spherical nanoparticle.

9. The nanoparticle bioconjugate of any one of claims 1 to 8, wherein the magnetic nanoparticle is selected from: an Fe₃O₄ (iron oxide) nanoparticle, an Fe(CO)₅ (iron pentacarbonyl) nanoparticle or an FeCl₂ (iron chloride) nanoparticle.

10. The nanoparticle bioconjugate of any one of claims 1 to 9, wherein the metallic nanoparticle is selected from: a gold (Au), a silver (Ag), platinum (Pt) or palladium (Pd), copper (Cu), nickel (Ni), cobalt (Co), or an alloy of two or more thereof or nanoparticle.

11. The nanoparticle bioconjugate of any one of claims 1 to 10, wherein the metallic nanoparticle is a gold or silver nanoparticle, preferably a gold nanoparticle.

12. The nanoparticle bioconjugate of any one of claims 1 to 11, wherein the nanoparticle bioconjugate is comprised of iron and gold nanoparticles, preferably wherein the bioconjugate comprises a central gold nanoparticle surrounded by 3-4 or more iron nanoparticles to form a flower-shaped nanoparticle.

13. The nanoparticle bioconjugate of any one of claims 1 to 12, wherein the nanoparticle bioconjugate is between about 5 nm to about 100 nm.

14. The nanoparticle bioconjugate of any one of claims 1 to 13, wherein the nanoparticle bioconjugate is between about 35 nm to 80 nm, preferably about 40 nm.

15. The nanoparticle bioconjugate of any one of claims 1 to 14, wherein the 1^{st} and 2^{nd} binding proteins are covalently conjugated to the 1^{st} and second nanoparticles.

16. The nanoparticle bioconjugate of any one of claims 1 to 15, wherein one or both the 1^{st} and 2^{nd} binding proteins are directly conjugated to the nanoparticles or conjugated via a linker moiety.

17. The nanoparticle bioconjugate of any one of claims 1 to 16, wherein the binding protein(s) is/are conjugated to the nanoparticle via a linker moiety, wherein the linker moiety is selected from a polyethylene glycol (PEG) moiety, an alkyne, an amine, an azide, biotin, a carboxyl, a hydroxyl, a methyl, maleimide, neutravidin, NHS, a hydrazone or a thiol linker group.

18. The nanoparticle bioconjugate of any one of claims 1 to 17, wherein the 1^{st} binding protein is any binding protein capable of specifically binding to a target moiety of a cancer cell.

19. The nanoparticle bioconjugate of any one of claims 1 to 18, wherein the binding protein for binding the target moiety on a cancer cell comprises, consists essentially of or consists of an antigen binding domain.

20. The nanoparticle bioconjugate of any one of claims 1 to 18, wherein the binding protein for binding the target moiety on a cancer cell comprises, consists essentially of or consists of a receptor or ligand, or part thereof, for binding the cancer cell.

21. The nanoparticle bioconjugate of any one of claims 1 to 20, wherein the target moiety on the cancer cell is selected from: a sugar, a lipid, a nucleic acid, a peptide or a protein.

22. The nanoparticle bioconjugate of any one of claims 1 to 21, wherein the cancer cell is any selected from: breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

23. The nanoparticle bioconjugate of any one of claims 1 to 22, wherein the target moiety on the cancer cell is an antigen of cancer cell selected from: HER2, EGFR, mesothelin, GPC3, MUC1, GD2, CEA, EpCAM, LeY, PCSA, CD19, CD20, Clec9a, CD276, PD-L1 and PD-L2.

24. The nanoparticle bioconjugate of any one of claims 1 to 23, wherein the antigen is HER2 and the cancer cell is any cancer cell overexpressing HER2 or is HER2 positive such as breast cancer or stomach cancer.

25. The nanoparticle bioconjugate of any one of claims 1 to 23, wherein the antigen is EGFR and the cancer cell is any cancer cell overexpressing epidermal growth factor receptor (EGFR) such as: a squamous cell carcinoma of head and neck (SCCHN), glioma, breast cancer, nasopharangeal cancer, or pancreatic cancer cell.

26. The nanoparticle bioconjugate of any one of claims 1 to 25, wherein the 2^{nd} binding protein is any binding protein capable of specifically binding to a target moiety of an immune cell.

27. The nanoparticle bioconjugate of any one of claims 1 to 26, wherein the immune cell is selected from: a T cell, an antigen presenting cell (APC), or a natural killer (NK) cell, preferably wherein the immune cell is a T cell.

28. The nanoparticle bioconjugate of any one of claims 1 to 27, wherein the target moiety on the immune cell is selected from: a sugar, a lipid, a nucleic acid, a peptide or a protein.

29. The nanoparticle bioconjugate of any one of claims 1 to 28, wherein the binding protein for binding the target moiety comprises, consists essentially of or consists of an antigen binding domain.

30. The nanoparticle bioconjugate of any one of claims 1 to 28, wherein the binding protein for binding the target moiety comprises, consists essentially of or consists of a receptor or ligand, or part thereof, for binding the immune cell.

31. The nanoparticle bioconjugate of any one of claims 1 to 30, wherein the immune cell is a T cell and the target moiety on the T cell that is bound by the 2^{nd} binding protein, is selected from: CD3, CD2, CD4, CD7, CD8, PD1, CTLA4, KIR, CD16, CD94, CD161, NTBA, CD19, CD20, CD22, CD30, CD33, CD38, CD40L, CD44, CD56, CD79b, CD80, CD86, CD135, CD137, CD138, CD154, EphA2, EGFR, and any combination thereof.

32. The nanoparticle bioconjugate of any one of claims 1 to 30, wherein the immune cell is an NK cell, and the binding protein is for binding an antigen selected from: KIR, CD16, CD94, CD 161 or CD56.

33. The nanoparticle bioconjugate of any one of claims 1 to 32, wherein one or both of the binding proteins are an antibody, or antigen binding fragment thereof.

34. The nanoparticle bioconjugate of any one of claims 1 to 33, wherein one or both of the binding proteins are antibodies selected from IgA, IgD, IgE, IgG, IgM, preferably wherein the antibody is an IgG.

35. The nanoparticle bioconjugate of any one of claims 1 to 33, wherein one or both of the binding proteins are antibody fragments selected from: a recombinant antibody fragment, a diabody, a triabody, a chimeric antibody, an F(ab′)2 fragment, an Fab′ fragment, an Fab′-SH fragment, a Fab fragment, an sFv fragment, a dsFv fragment, a bispecific sFv fragment, a bispecific dsFv fragment, a single chain Fv protein (scFv), a disulfide stabilized Fv protein, or a combination thereof.

36. The nanoparticle bioconjugate of any one of claims 1 to 35, wherein the 1^{st} and 2^{nd} binding proteins are the same antibody format.

37. The nanoparticle bioconjugate of any one of claims 1 to 36, wherein the bioconjugate further comprises a cell penetrating peptide.

38. A pharmaceutical composition comprising any nanoparticle bioconjugate of any one of claims 1 to 37, optionally in combination with a pharmaceutically acceptable excipient or carrier.

39. A method of treating or preventing cancer in a subject, the method comprising administering to a subject in need thereof, a nanoparticle bioconjugate of any one of claims 1 to 37, or pharmaceutical composition of claim 38.

40. Use of a nanoparticle bioconjugate of any one of claims 1 to 37, in the manufacture of a medicament for the treatment or prevention of cancer.

41. A nanoparticle bioconjugate of any one of claims 1 to 37, or a pharmaceutical composition of claim 38, for use in the treatment or prevention of cancer.
